(19) United States
(12) Patent Application Publication
(10) Pub. No.: US 2010/0222728 A1
(43) Pub. Date: Sep. 2, 2010

(54) Body Pad

(75) Inventor: Jeffrey S. Brooks, St. Louis, MO (US)

Correspondence Address: SENNIGER POWERS LLP, 100 NORTH BROADWAY, 17TH FLOOR, ST LOUIS, MO, 63102, US

(73) Assignee: DR. BROOKS INNOVATIONS, LLC, St. Louis, MO (US)

(21) Appl. No.: 12/680,922

(22) PCT Filed: Oct. 3, 2008
(86) PCT No.: PCT/US08/78680

§ 371 Date: Mar. 31, 2010

Related U.S. Application Data
(60) Provisional application No. 60/977,828, filed on Oct. 5, 2007.
(60) Provisional application No. 61/048,325, filed on Apr. 28, 2008.

(51) Int. Cl. A61F 5/01

(52) U.S. Cl. 602/30

(57) ABSTRACT

A body pad for application to the skin for treating an ailment. The pad includes a pad body, a recess in the pad body extending from the outer surface of the pad body toward the inner surface a distance which is less than an overall thickness of the body, and a thin, flexible wall at a bottom of the recess. The wall is adapted to deform outwardly into the recess to provide a cavity between the pad body and the skin for holding a volume of medication which has been pre-applied to the skin. Other pad features are disclosed.

### FIELD OF THE INVENTION

The present invention generally relates to pads for placement on the body for protection against injury or for treatment of an ailment, especially (but not exclusively) foot ailments.

### BACKGROUND OF THE INVENTION

Conventional foot pads have various drawbacks. They are often difficult to use and keep in place when applied over medications (e.g., gels or creams). Further, they are typically not removable, readily cleaned and reusable on a daily basis. There is a need, therefore, for an improved line of foot pads.

### SUMMARY OF THE INVENTION

This invention is directed to body pads having one or more of the features herein.

In one embodiment, a body pad of this invention is applied to the skin for treating an ailment. The pad comprises a pad body having an inner surface which is adapted for contact with the skin and an outer surface which faces away from the skin when in use. A recess in the pad body extends from the outer surface of the pad body toward the inner surface a distance which is less than an overall thickness of the body. A thin, flexible wall is provided adjacent the bottom of the recess. This wall is adapted to deform outwardly into the recess to provide a cavity between the pad body and the skin for holding a volume of medication which has been pre-applied to the skin.

In another embodiment, this invention is directed to a toe spacer comprising an elongate pad body, and a hinge extending transversely of the pad about midway between opposite ends of the body. The pad body has curved surfaces on opposite sides of the hinge whereby when the pad body is folded about the hinge the curved surfaces are adapted for contact with one another when the pad is placed between two toes.

In another embodiment, this invention is directed to a toe crest pad comprising a pad body having a proximal end, a distal end, opposite sides, and an opening in the pad body sized for receiving one or more toes. The pad body has a distal end margin forming an anti-flex distal portion extending from adjacent one side of the pad body to adjacent the opposite side of the pad body. The anti-flex distal portion has a top face for contact with the one or more toes at the bottom of the foot when the pad is applied to the foot. The pad body has a proximal end margin forming an anti-flex proximal portion extending from adjacent one side of the body to adjacent the opposite side of the pad body. The anti-flex proximal portion has a bottom face for contact with the toes or foot at the top of the foot when the pad is applied to the foot.

In another embodiment, this invention is directed to a bi-plane pad comprising a pad body having a longitudinal axis and a transverse axis. The aid pad body having an inner surface for contact with the skin and an outer surface. A recess in the inner surface extends transversely of the pad. The inner surface has side regions on opposite sides of the recess. The recess has a width along the longitudinal axis of the pad body sufficient for relieving pressure on one or more metatarsal heads of the foot or a vertical boney prominence on the back of a heel of a foot when the pad is applied to the foot.

In another embodiment, this invention is directed to a heel lift comprising a lift body having a longitudinal axis, an inner surface for contact with the foot or a sock on the foot, and an outer surface for contact with footwear worn on the foot. The inner surface has side regions on opposite sides of the recess. A U-shaped recess in the inner surface extends along the longitudinal axis for receiving a central portion of the heel of the foot, portions of the foot on opposite sides of the central portion being supported by the side regions of the inner surface of the lift body.

In another embodiment, this invention is directed to a toe crest pad comprising an elongate pad body having a substantially planar inner surface and an outer surface. A hinge extends transversely of the pad at a location between opposite ends of the body. First and second openings in the pad body on opposite sides of the hinge are adapted to align with one another when the pad body is folded about the hinge to a folded condition for application to a foot. First and second ribs on the outer surface of the pad body extend transversely of the pad body immediately adjacent the hinge on opposite sides of the hinge and between the first and second openings. The first and second ribs having generally flat faces which combine to provide a substantially flat surface when the pad is in said folded condition. A third rib extends transversely of the pad body on the outside surface of the body adjacent a proximal end of the pad body. The third rib has a generally semi-circular transverse cross section with a curved outer surface. The pad is adapted to be placed on the foot with one or more toes received in said aligned openings, with the first and second ribs positioned on the underside of the foot with their flat faces combining to provide a substantially flat downwardly facing surface, and with the third rib positioned on the upper side of the foot with its curved outer surface facing upward.

### BRIEF DESCRIPTION OF THE DRAWINGS

FIG. 1 is a perspective view of one embodiment of an all-purpose body pad of this invention;

FIG. 2 is a front view of the pad of FIG. 1;

FIG. 3 is a side view of the pad of FIG. 1;

FIG. 4 is an enlarged section taken on line 4-4 of 2;

FIG. 4A is a view similar to FIG. 4 but showing medication in a pocket of the pad;

FIG. 5 is a top view of the pad of FIG. 1;

FIG. 6 is a section taken on line 6-6 of FIG. 2;

FIG. 6A is a view of a pad having a peel-off backing sheet;

FIGS. 7, 8 and 8A or views similar to FIGS. 2, 4 and 4A but showing a different embodiment of the all-purpose pad;

FIG. 9 is perspective view of one embodiment of a metatarsal pad of this invention;

FIG. 10 is a front view of the pad of FIG. 9;

FIG. 11 is a side view of the pad of FIG. 9;

FIG. 12 is a section taken on line 12-12 of FIG. 10;

FIG. 13 is a top view of the pad of FIG. 9;

FIG. 14 is a view similar to FIG. 10 but with a pocket formed in the pad for receiving medication;

FIG. 15 is a section taken on line 15-15 of FIG. 14;

FIGS. 16-18 are drawings illustrating how the pad of FIG. 9 may be positioned on a foot;

FIG. 19 is a perspective view of one embodiment of a bunion-sesamoid pad of this invention;

FIG. 20 is a front view of the pad of FIG. 19;

FIG. 21 is a side view of the pad of FIG. 19;

FIG. 22 is a top view of the pad of FIG. 19;

FIGS. 23-24 are drawings illustrating how the pad of FIG. 19 may be positioned on a foot;

FIG. 25 is a perspective view of one embodiment of a corn pad of this invention;

FIG. 26 is a front view of the pad of FIG. 25;

FIG. 27 is a side view of the pad of FIG. 25;

FIG. 28 is a top view of the pad of FIG. 25;

FIG. 29 is a front view of the pad of FIG. 25 but with a pocket formed in the pad for receiving medication;

FIG. 30 is a section taken on line 30-30 of FIG. 29;

FIGS. 30 and 31 are drawings illustrating how the pad of FIG. 25 may be positioned on a foot;

FIG. 33 is a perspective view of one embodiment of a toe straightening pad of this invention;

FIG. 33A is a front view of the pad of FIG. 33;

FIG. 34 is a top view of the pad of FIG. 33;

FIG. 35 is a side elevation of the pad of FIG. 33;

FIG. 36 is a front view of the pad of FIG. 33 but with a pocket formed in the pad for receiving medication;

FIG. 37 is a section taken on line 37-37 of FIG. 36;

FIGS. 38 and 39 are drawings illustrating how the pad of FIG. 32 may be positioned on a foot;

FIG. 40 is a perspective view of one embodiment of a toe spacer pad of this invention;

FIG. 41 is a top view of the pad of FIG. 40;

FIG. 42 is a front view of the pad of FIG. 40;

FIG. 43 is an end view of the pad of FIG. 40;

FIG. 44 is a view illustrating the pad of FIG. 40 bent about a hinge for placement between two toes;

FIG. 45 is a side view of the pad of FIG. 44;

FIG. 46 is a front view of the pad of FIG. 44;

FIG. 47 is a top view of the pad of FIG. 44;

FIGS. 48 and 49 are drawings illustrating how the pad of FIG. 44 may be positioned between two toes of a foot;

FIG. 50 is a perspective view of one embodiment of a three-toe crest pad of this invention;

FIG. 51 is a front view of the pad of FIG. 50;

FIG. 52 is a top view of the pad of FIG. 50;

FIG. 53 is a side view of the pad of FIG. 50;

FIG. 54 is a perspective view of the pad of FIG. 50 in a folded condition for placement on a foot;

FIG. 55 is a front view of the folded pad of FIG. 54;

FIG. 56 is a top view of the folded pad of FIG. 54;

FIG. 57 is a side view of the folded pad of FIG. 54;

FIGS. 58 and 59 are views showing the folded pad of FIG. 54 positioned on a foot;

FIG. 60 is a perspective view of one embodiment of a two-toe crest pad of this invention;

FIG. 61 is a front view of the pad of FIG. 60;

FIG. 62 is a top view of the pad of FIG. 60;

FIG. 63 is a side view of the pad of FIG. 60;

FIG. 64 is a perspective view of the pad of FIG. 60 in a folded condition for placement on a foot;

FIG. 65 is a front view of the folded pad of FIG. 64;

FIG. 66 is a top view of the folded pad of FIG. 64;

FIG. 67 is a side view of the folded pad of FIG. 64;

FIGS. 68 and 69 are views showing the folded pad of FIG. 64 positioned on a foot;

FIG. 70 is a perspective view of one embodiment of a one-toe crest pad of this invention;

FIG. 71 is a front view of the pad of FIG. 70;

FIG. 72 is a top view of the pad of FIG. 70;

FIG. 73 is a side view of the pad of FIG. 70;

FIG. 74 is a perspective view of the pad of FIG. 70 in a folded condition for placement on a foot;

FIG. 75 is a front view of the folded pad of FIG. 74;

FIG. 76 is a top view of the folded pad of FIG. 74;

FIG. 77 is a side view of the folded pad of FIG. 74;

FIGS. 78 and 79 are views showing the folded pad of FIG. 74 positioned on a foot;

FIG. 80 is a perspective view of one embodiment of a pan metatarsal pad of this invention;

FIG. 81 is a front view of the pad of FIG. 80;

FIG. 82 is a side view of the pad of FIG. 80;

FIG. 83 is a top view of the pad of FIG. 80;

FIG. 84 is a perspective view of one embodiment of a pinch callous pad of this invention;

FIG. 85 is a front view of the pad of FIG. 84;

FIG. 86 is a side view of the pad of FIG. 84;

FIG. 87 is a top view of the pad of FIG. 84;

FIGS. 88 and 89 are drawings illustrating how the pad of FIG. 84 may be positioned on a foot;

FIG. 90 is a perspective view of one embodiment of a bi-plane pad of this invention;

FIG. 91 is a front view of the pad of FIG. 90;

FIG. 92 is a top view of the pad of FIG. 90;

FIG. 93 is a side view of the pad of FIG. 90;

FIGS. 94-96 are drawings illustrating examples of how the pad of FIG. 90 may be positioned on a foot and in footwear;

FIG. 97 is a front elevation of one embodiment of a heel lift of this invention;

FIGS. 98 and 99 illustrate how the heel lift of FIG. 97 may be used;

FIG. 100 is a perspective view of one embodiment of a corn hammertoe pad of this invention;

FIG. 101 is a front view of the pad of FIG. 100;

FIG. 102 is a side view of the pad of FIG. 100;

FIG. 103 is a front view similar to FIG. 101 but with a thin wall of the pad body deflected to form a cavity to receive medication;

FIG. 104 is a section taken on line 104-104 of FIG. 103;

FIG. 105 is a top view of the pad of FIG. 100

FIG. 106 illustrates how the pad of FIG. 100 may be used;

FIG. 107 is a perspective view of another embodiment of a three-toe crest pad of this invention;

FIG. 108 is front view of the pad of FIG. 107;

FIG. 109 is a bottom view of the pad of FIG. 107;

FIG. 110 is a side view of the pad of FIG. 107;

FIGS. 111-113 are drawings illustrating examples of how the pad of FIG. 107 may be positioned on a foot;

FIG. 114 is a perspective view of another embodiment of a two-toe crest pad of this invention;

FIG. 115 is front view of the pad of FIG. 114;

FIG. 116 is a bottom view of the pad of FIG. 114;

FIG. 117 is a side view of the pad of FIG. 114;

FIGS. 118-120 are drawings illustrating examples of how the pad of FIG. 114 may be positioned on a foot and in footwear;

FIG. 121 is a perspective view of another embodiment of a one-toe crest pad of this invention;

FIG. 122 is front view of the pad of FIG. 121;

FIG. 123 is a bottom view of the pad of FIG. 121;

FIG. 124 is a side view of the pad of FIG. 121;

FIGS. 125-127 are drawings illustrating examples of how the pad of FIG. 121 may be positioned on a foot and in footwear;

FIG. 128 is a perspective view of one embodiment of a bunion toe spacer pad of this invention;

FIG. 129 is front view of the pad of FIG. 128;

FIG. 130 is a side view of the pad of FIG. 128;

FIG. 131 is a top view of the pad of FIG. 128;

FIG. 132 is a drawing illustrating how the pad of FIG. 128 may be positioned on a foot.

Corresponding reference characters indicate corresponding parts throughout the drawings.

### DESCRIPTION OF THE PREFERRED EMBODIMENTS

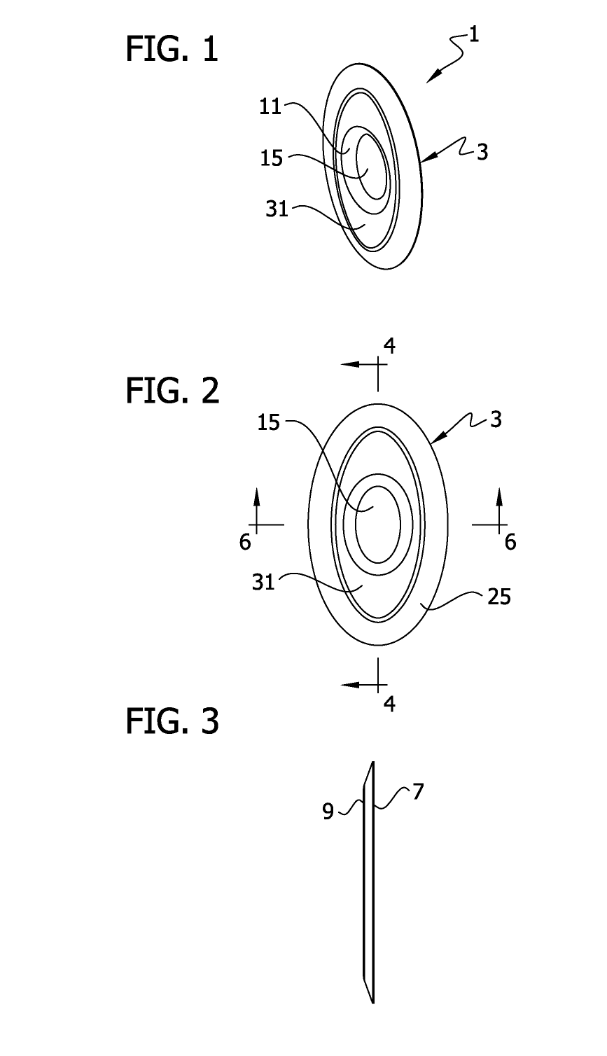

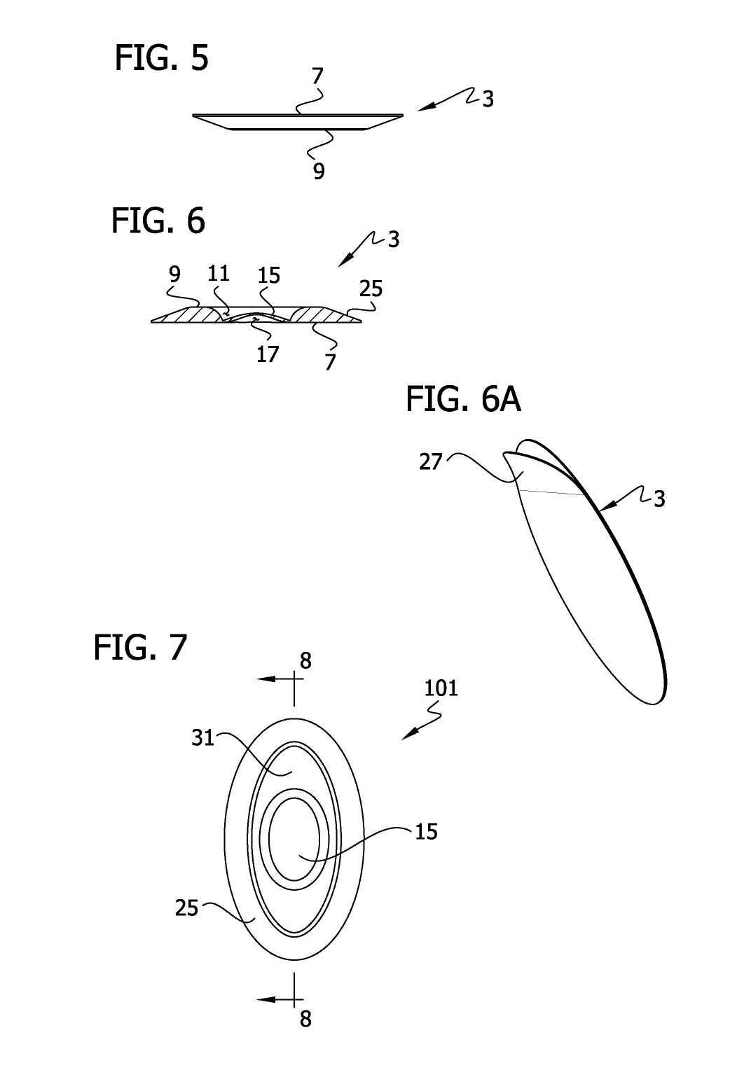

Referring now to the drawings, FIGS. 1-6 show one-embodiment of an all-purpose pad 1 incorporating features of this invention. By way of example but not limitation, the pad may be used to treat bunions on the feet. The pad 1 comprises a relatively thin, oval-shaped body 3 of a soft, pliable, gel-like material (e.g., polyurethane) which is sufficiently pliable and flexible to mold itself to the contours of the body when applied to the skin. In one embodiment, the pad body has a Shore OO hardness of about 45-70 (+/−7). The material is desirably substantially transparent (so that it is not readily visible on the skin), non-toxic when applied to the skin, hypoallergenic and water resistant. One suitable polyurethane material for use in making the pad body is commercially available from PolyWorks Inc. (24 Albion Road, Suite 101, Lincoln, RI 02865.

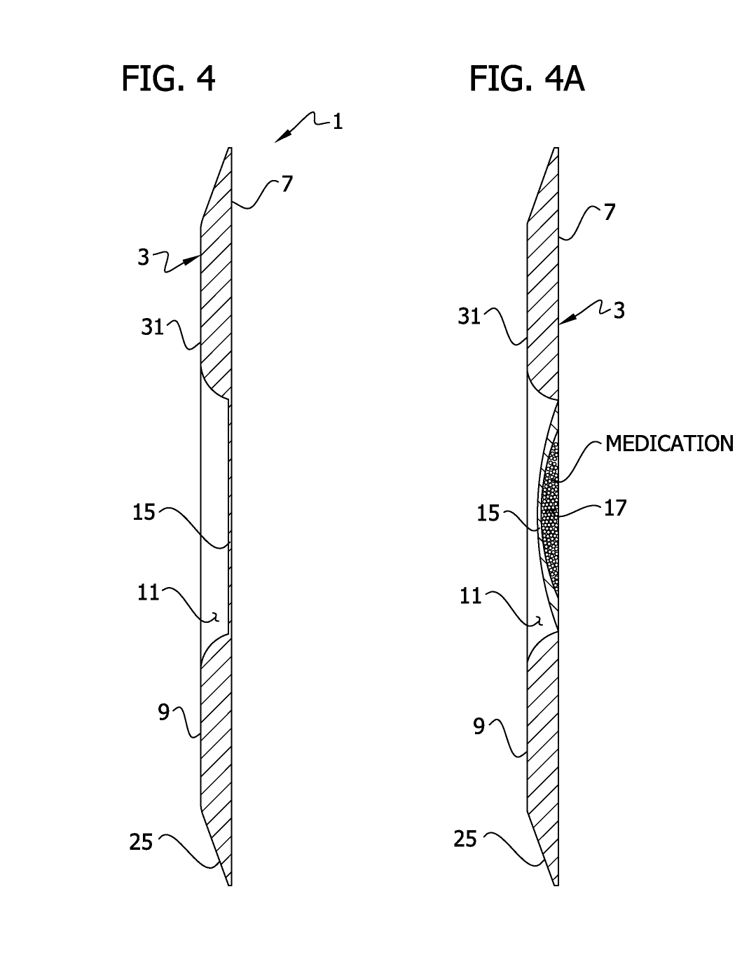

The body 3 of the pad 1 has an inner surface 7 which is adapted for contact with the skin and an outer surface 9 which faces away from the skin when in use. A recess 11 extends from the outer surface of the pad body toward the inner surface a distance which is less than the overall thickness of the body. As a result, a thin, flexible wall 15 is provided at the bottom of the recess (FIG. 4). The wall is adapted to deform outwardly into the recess above it to provide what may be referred to as a pocket, cavity or volume 17 between the pad body and the skin for holding a small quantity or volume of medication which has been pre-applied to the skin (see FIG. 4A). Again, by way of example but not limitation, the maximum thickness of the pad from its inner surface 7 to its outer surface 9 may be about 0.100 in. and the thin wall 15 may have a thickness of about 0.010 in. These dimensions may vary. In the particular embodiment of FIG. 1, the thin wall 15 is formed as an integral part of the pad body 3, and the bottom surface of the thin wall is generally co-planar with the surrounding portion of the inner surface 7 of the pad body 3. However, it will be understood that the thin wall 15 could be formed as a part separate from the rest of the pad body 3 and then secured in place over the recess. The recess 11 is generally oval in shape, but this shape may vary. The peripheral margin 25 of the pad is skived or tapered from the outer surface to the inner surface and provides additional area at the inner surface of the pad body for adhesively securing the pad in place on the skin.

The inner surface 7 of the pad body 3 has an adhesive coating for adhesion to the skin. A protective peel-off backing sheet 27 (similar to that shown in FIG. 6A) is provided to shield the adhesive coating until the pad is ready for use, at which time the backing layer is removed to expose the adhesive for application of the pad to the skin. The backing sheet 27 is of a relatively stiff, clear material (e.g., 2.0 mil Mylar film) having a release coating thereon to facilitate ready removal of the pad body 3 from the backing sheet.

The outer surface 9 of the pad body 3 comprises a thin layer of antibacterial material. By way of example, this layer may be formed by a 0.75 mil-thick polyurethane film impregnated with silver. The central portion of the pad body 3 surrounding the recess 11 has a desired overall thickness in the range of about 0.075 in. to about 0.125 in. (e.g., 0.10 in.).

In use, the skin is cleaned and dried, and a volume of medication is applied to the skin over an ailment being treated (e.g., a bunion). The backing layer 27 is removed from the pad, and the pad body 3 is placed, inner surface down, on the skin with the thin wall 15 and recess 11 generally aligned with the medication. The pad is gently stretched and molded to the skin. During the application process, the thin wall 15 of the pad body deflects outward into the recess 11, i.e., toward the upper surface of the pad body, to create the pocket 17 for receiving and holding a quantity of medication captive at the desired location on the skin. (The deflected position of the wall 15 is illustrated in FIG. 4A which shows the pad applied to skin with a quantity of medication received in the pocket 17.) Further, where the pad 1 s applied to the foot and the foot is placed in footwear, the pressure applied to the pad by the footwear will be borne by the outer surface 9 of the pad surrounding the recess 11, thus relieving the area of the foot below the recess and thin wall from pressure which might otherwise cause pain and/or inhibit treatment of the malady. The portion 31 of the outer surface surrounding the recess and bearing the load on the pad may be referred to as the “pressure ridge.”

At night, before bed, the pad is removed and placed, inner surface 7 down, on a pad storage sheet of a suitable material (e.g., Silicone laminate). The pad can be re-applied in the morning. The pad can be cleaned, as needed, by washing it with soap and water and allowing the pad to air dry before it is re-applied. Washing automatically restores the adhesive characteristics of the inner surface 7 of the pad body 3.

The size and shape of the pad 1 will vary. In general, the recess 11 in the pad body 3 should be sized sufficient to receive and hold a desired volume of medication. By way of example but not limitation, the area of the thin wall 15 at the bottom of the recess 11 may be in the range of 0.10-0.01 in², and the thin wall is deformable to form a cavity 17 having a volume or capacity in the range of about 0.01 to 5.75 in³. (The numbers can vary.) The peripheral margin 25 of the pad body 3 should be sufficiently large to insure that the pad remains adhered to the skin during use. Desirably, but not necessarily, the pad is symmetrical about both its longitudinal and transverse central axes.

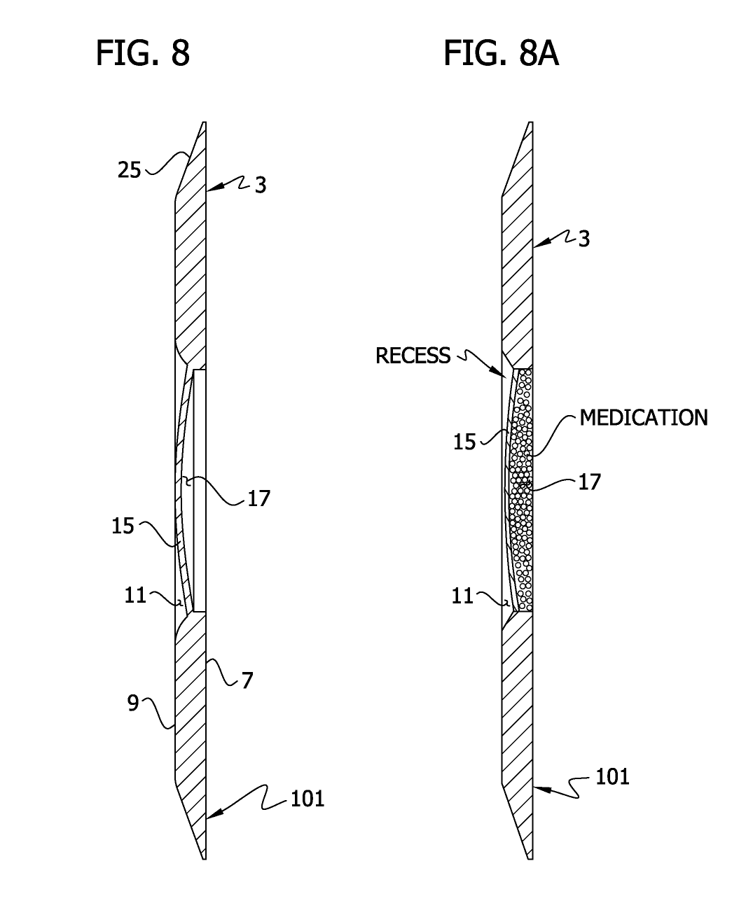

FIGS. 7, 8 and 8A show a second embodiment of a pad of this invention, generally designated 101. The pad is substantially identical to the pad 1 and corresponding parts are designated by corresponding reference numbers. The main difference is that the recess 11 extends from the outer surface 9 of the pad body 3 to the inner surface of the pad body, and the thin wall 15 is positioned at a location spaced from both surfaces, e.g., generally midway between the surfaces. This location has the advantage of providing a larger cavity 17 for receiving a larger volume of medication (see FIG. 8A).

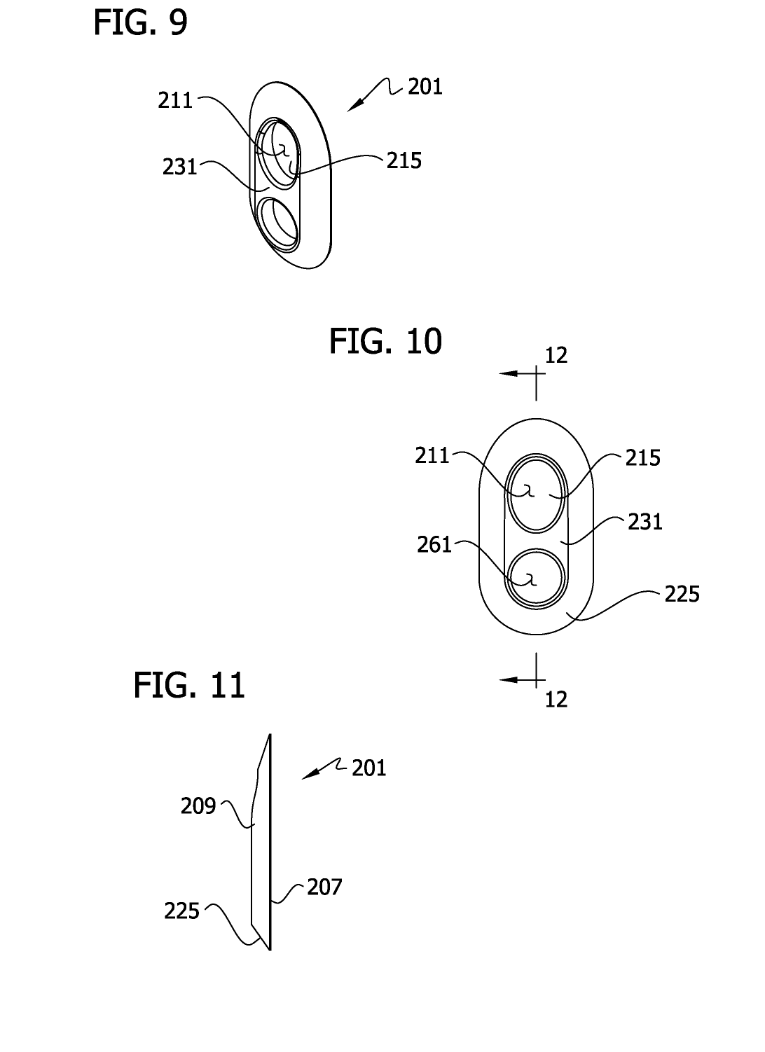

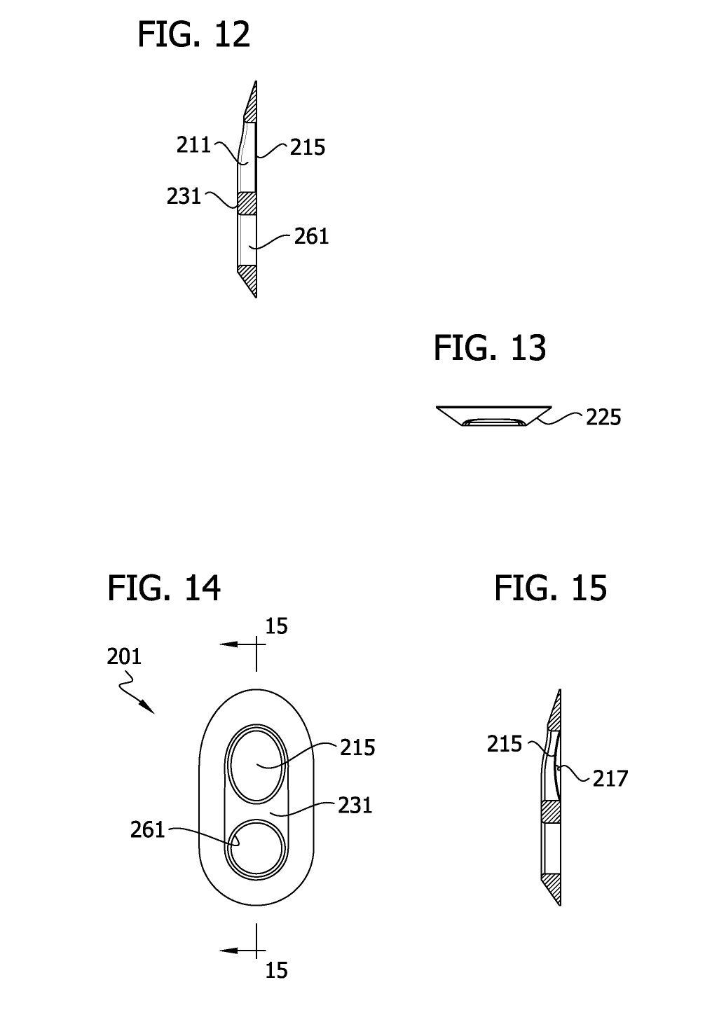

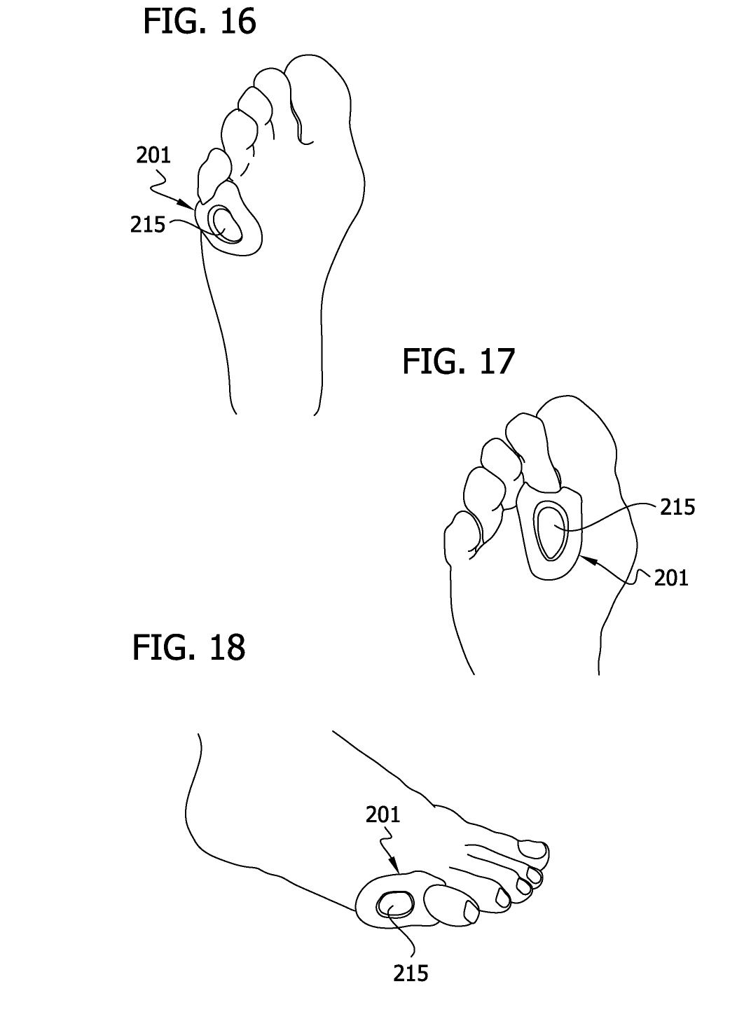

FIGS. 9-15 illustrate one embodiment of a lesser metatarsal pad, generally designated 201, for application to the foot as shown in the photos of FIGS. 16-18. This pad 201 is constructed in much the same manner as the all-purpose pad 1 described above, and corresponding parts are indicated by corresponding reference numbers plus 200. The pad 201 comprises a pad body 203 having a recess 211 and thin wall 215 adjacent the proximal end of the pad. The pad also has an adhesive and backing layer on its inner surface 207 and an antibacterial coating on its outer surface, as previously described. Further, the pad body has an opening 261 through it toward the distal end of the pad for receiving a digit such as a toe for enabling the pad to be positioned as shown in FIGS. 16-18. (As used herein, “proximal” means in the direction toward the head and “distal” means in a direction away from the head.) The central portion of the pad body 203 surrounding the recess 211 has a desired thickness in the range of about 0.1 in. to about 0.2 in. (e.g., 0.187 in.).

To use the pad 201, a toe is inserted through the toe opening 261 and the thin wall 215 of the recess 211 is positioned over the appropriate metatarsal. For example, in FIG. 16 the fifth (little) toe is received in the toe opening 261 and the thin wall of the recess is positioned over the fifth metatarsal. In FIG. 17, the second toe is received in the opening 261 and the thin wall 215 of the recess is positioned over the second metatarsal. In FIG. 18, the fifth (little) toe is received in the opening and the thin wall of the recess is positioned alongside the foot over a tailor's bunion. As in the previous pads 1, 101, the pad 201 has a pressure relief ridge 231 and a tapered peripheral margin 225 which functions as a securing flap for providing additional area to adhere the pad body securely to the foot.

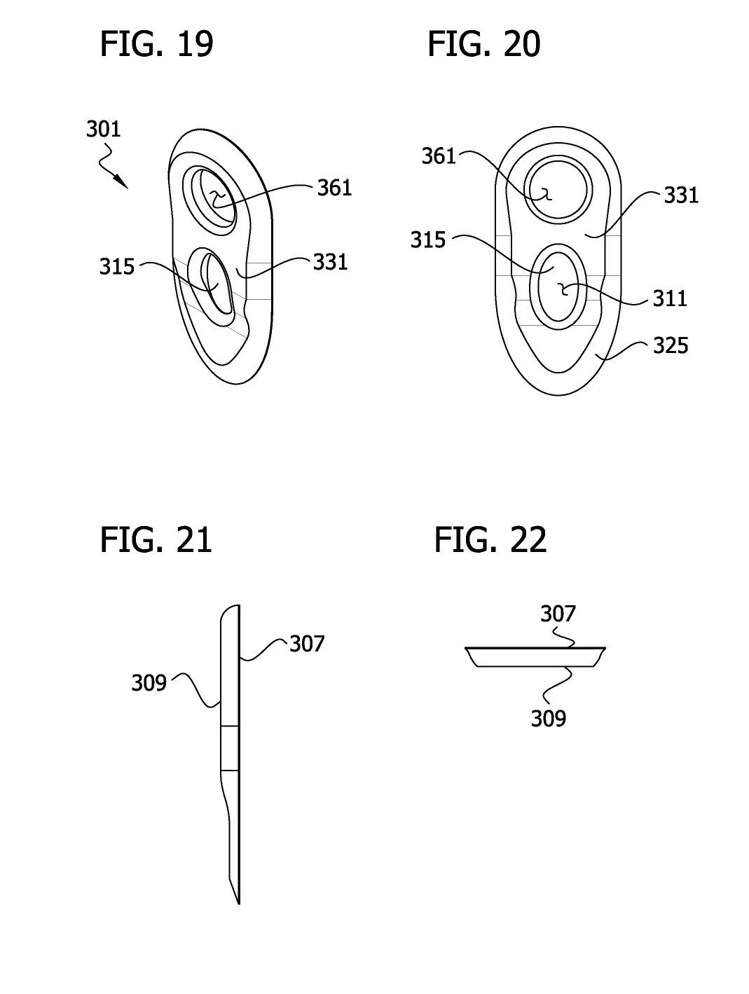

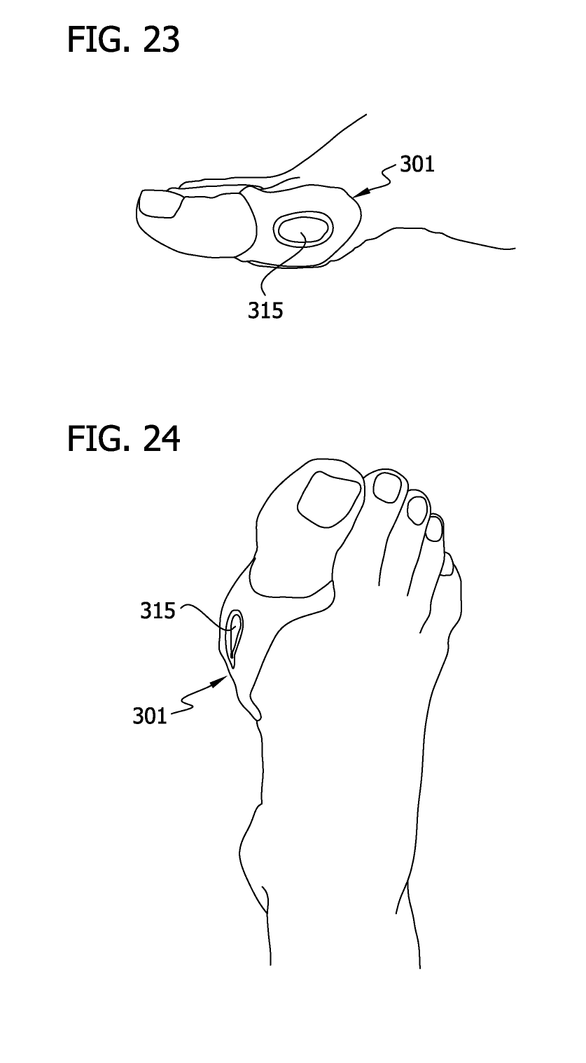

FIGS. 19-22 illustrate a bunion/sesamoid pad, generally designated 301, for application to the foot as shown in the photos of FIGS. 23 and 24. This pad is constructed in much the same manner as the pad 1 described above, and corresponding parts are indicated by corresponding reference numbers plus 300. The pad 301 comprises a pad body 303 having a recess 311 and thin wall 315 adjacent the proximal end of the pad. The pad also has an adhesive and backing layer on its inner surface and an antibacterial coating on its outer surface, as previously described. Further, the pad body has an opening 361 through it toward the distal end of the pad for receiving a digit such as a toe for enabling the pad to be positioned as shown in the photos. (As used herein, “proximal” means in the direction toward the head and “distal” means in a direction away from the head.) The central portion of the pad body 303 surrounding the recess 311 has a desired thickness in the range of about 0.1 in. to about 0.2 in. (e.g., 0.187 in.).

To use the pad 301 (see FIGS. 23 and 24), the first (big) toe is of the foot is inserted through the toe opening 361 and the thin wall 315 of the recess is positioned alongside the foot over a bunion. Similar to the pads of the previous embodiments, the thin wall 315 is capable of deflecting into the recess 315 to form a cavity (not shown) to receive a quantity of medication on the foot, if such medication is used. In FIG. 24, the first (big) toe is received in the opening and the thin wall 315 of the recess 311 is positioned over the lateral sesamoid and/or medial sesamoid, as needed to relieve pain. As in the previous pads 1, 101, 201, the pad 301 has a pressure relief ridge 331 and a tapered peripheral margin which functions as a securing flap for providing additional area to adhere the pad body securely to the foot.

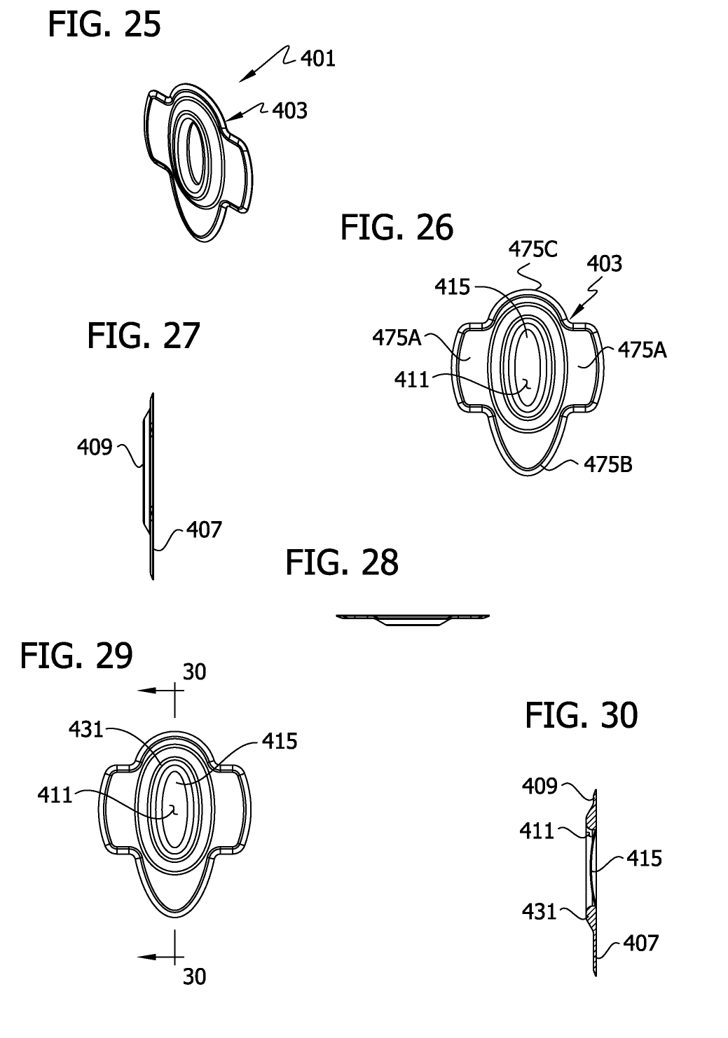

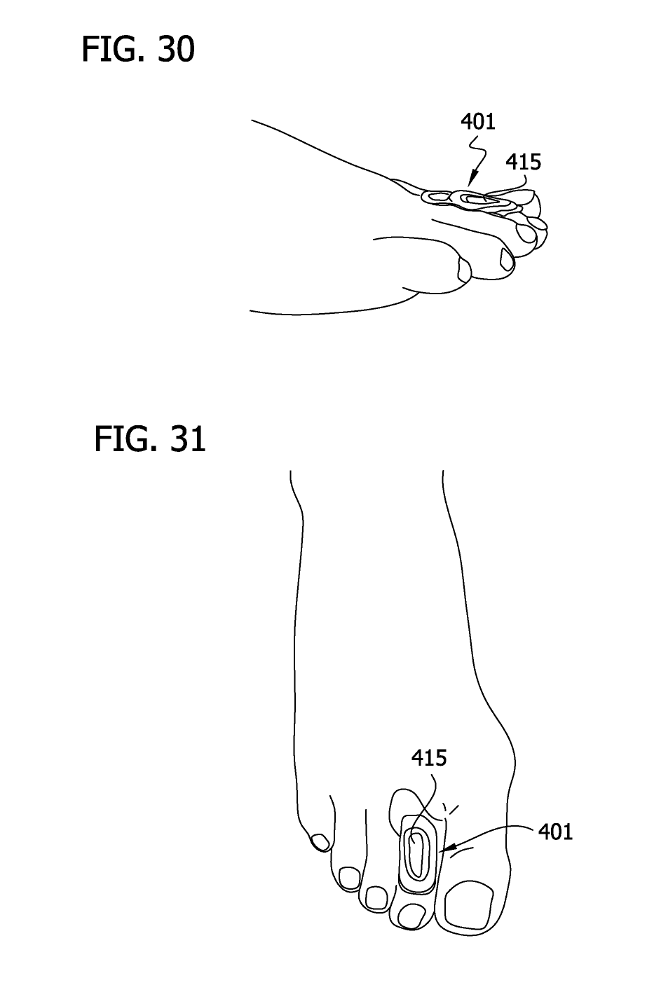

FIGS. 25-30 illustrate one embodiment of a corn pad, generally designated 401, for application to the foot as shown in FIGS. 31 and 32. This pad is constructed in much the same manner as the all-purpose pad 1 described above, and corresponding parts are indicated by corresponding reference numbers plus 400. The pad body 403 has a recess 411 and thin wall 415 located generally centrally of the pad for creating a cavity to receive medication, if needed, as previously described with respect to the pad 1. The pad also has an adhesive and backing layer on its inner surface 407 and an antibacterial coating on its outer surface 409, as previously described. The peripheral margin of the pad comprises a plurality of securing flaps 475 for adhesively securing the pad body 403 to the skin. More specifically, in one embodiment, four such flaps are provided, two relatively large lateral flaps 475A at opposite sides of the pad body 403, a relatively large proximal flap 475B at the proximal end of the pad body, and a smaller distal flap 475C at the distal end of the pad body. The central portion of the pad body 403 surrounding the recess 411 has a desired thickness in the range of about 0.075 in. to about 0.125 in. (e.g., about 0.10 in.).

In use, the pad body 403 of the pad 401 is positioned on the skin with the recess 411 and thin wall 415 which aligns with a corn on top of a toe of the foot (see FIGS. 31 and 32). If medication is used, a quantity of the medication is received in the cavity created by the deflection of the thin wall 415 into the recess 411. As in the previous pads 1, 101, 201, 301, the pad 401 has a pressure relief ridge 431.

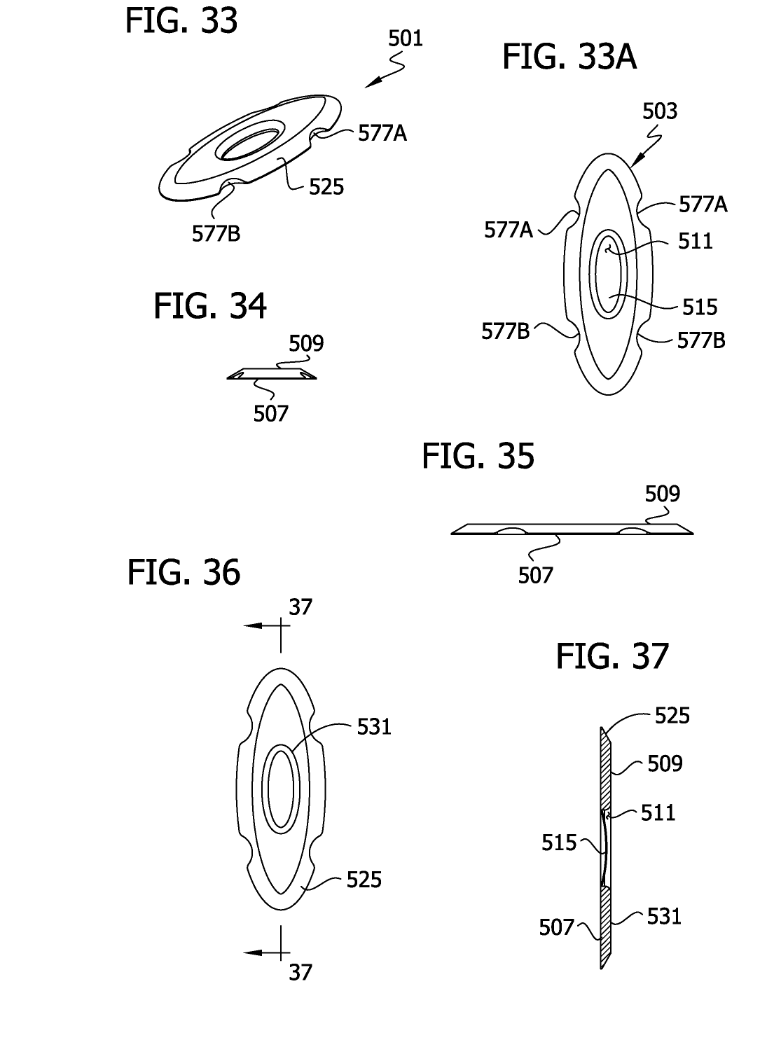

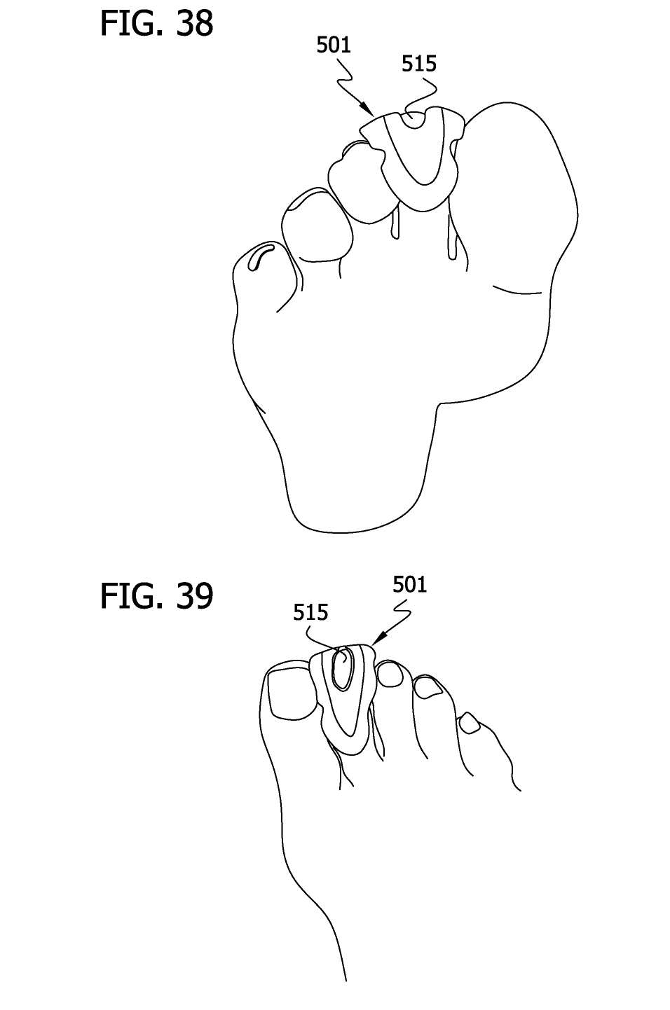

FIGS. 33-37 illustrate one embodiment of a toe straightening pad, generally designated 501, for application to the foot as shown in FIGS. 38 and 39. This pad is constructed in much the same manner as the all-purpose pad 1 described above, and corresponding parts are indicated by corresponding reference numbers plus 500. The pad is elongate and has a recess 511 and a thin wall 515 located generally centrally of the pad. The pad also has an adhesive and backing layer on its inner surface 507 and an antibacterial coating on its outer surface 509, as previously described. The peripheral margin 525 of the pad is skived and is provided with areas 577 of weakness (FIG. 33) to facilitate stretching of the pad body generally in the direction of the central longitudinal axis of the pad body, i.e., the central axis running from the distal end of the pad to the proximal end of the pad. In the illustrated embodiment, four such areas 577 of weakness are illustrated. These areas comprise two notches 577A in the peripheral margin of the pad body 503 toward the distal end of the pad body at opposite sides of the body, and two notches 577B in the peripheral margin of the pad body toward the proximal end of the pad body at opposite sides of the body. The central portion of the pad body 503 surrounding the recess 511 has a desired thickness in the range of about 0.075 in. to about 0.125 in. (e.g., about 0.10 in.).

The pad 501 is used to apply a straightening force to a toe which is bent (e.g., a mallet toe). Toes afflicted with this ailment often have bunions at their outer ends which require treatment. In use, the pad body 503 is positioned on the skin with the distal end of the pad body adhered to the underside of a toe at the distal phalangeal joint of the toe, and then pulled and stretched over the tip of the toe and back onto the top of the toe with a force sufficient to straighten the toe (see FIG. 38). The proximal end of the pad is then adhered to the skin on top of the toe generally at the proximal phalangeal joint to secure the pad in place with the toe straight (see FIG. 39). In this position, the thin wall 515 and recess 511 of the pad body 503 are located such that they are aligned with the outer tip of the toe to overlie medication on any bunion which may be present. If medication is used, a quantity of the medication is received in the cavity created by the deflection of the thin wall 515 into the recess 511. As in the previous pads 1, 101, 301, 401, the pad 501 has a pressure relief ridge 531.

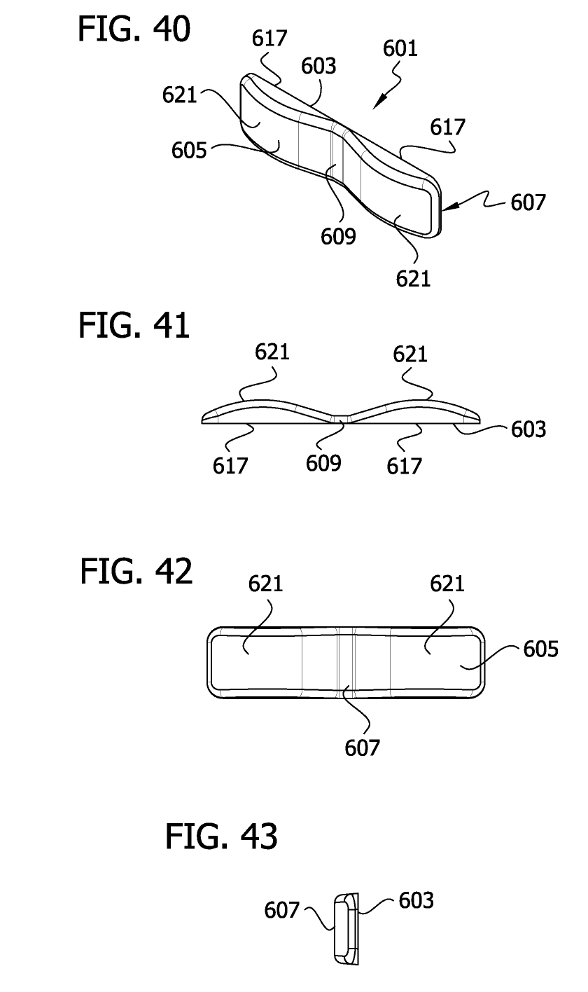

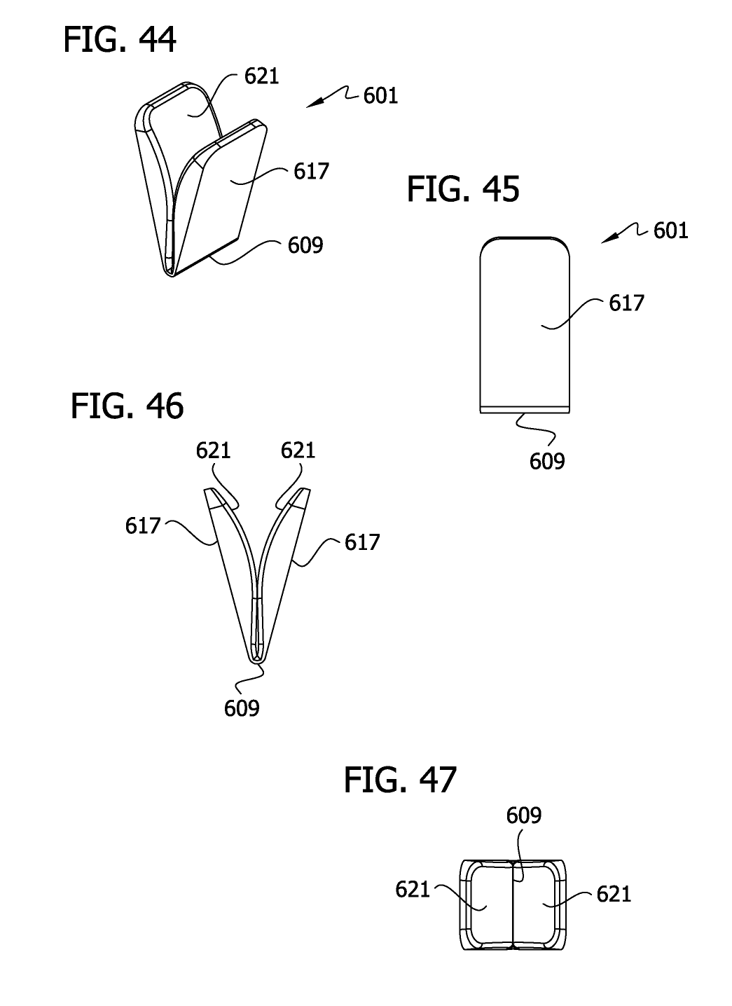

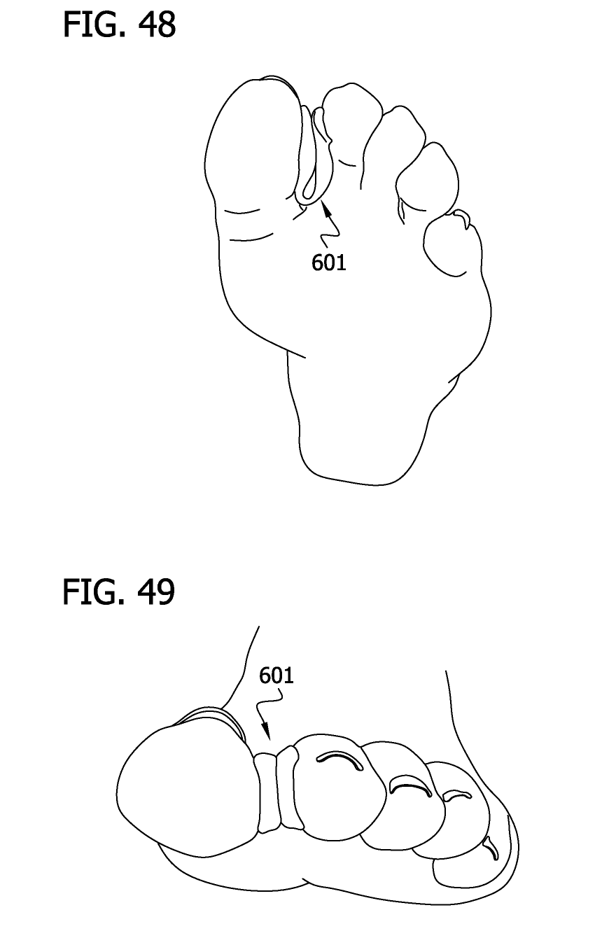

FIGS. 40-47 illustrate one embodiment of a toe spacer, generally designated 601, for application to the foot as shown in FIGS. 48 and 49. This pad is constructed of the same materials as the all-purpose pad 1 described above. The pad 601 also has an adhesive and backing layer on its inner surface 603 and an antibacterial coating on its outer surface 605, as previously described. However, unlike the previous pads, the pad 601 does not have a recess and thin wall. The pad comprises an elongate, generally rectangular pad body 607 having a line of weakness providing a hinge 609 (see FIGS. 40 and 44) extending generally transverse to the longitudinal axis of the body, preferably about midway between the ends of the body. The inner surface 603 of the pad body 607 comprises left and right substantially planar portions 617 on opposite sides of the hinge 609. The outer surface 605 of the pad body comprises left and right curved portions 621 on opposite sides of the hinge 609. The maximum thickness of each of the left and right curved portions 621 is desirably in the range of about 0.1 in. to about 0.15 in. (e.g., about 0.125 in.) for a pad 601 to be inserted between the first (big) toe and the second toe, and desirably in the range of about 0.05 in. to about 0.075 in. (e.g., about 0.062 in.) for a pad 601 to be inserted between any two of the lesser (second, third, fourth and fifth) toes.

In use, the backing layer is removed from the inner surface 603 of the pad body 607, and the pad body 607 is then bent in half about the hinge 609 to the position illustrated in FIGS. 44 and 46. In this position the planar inner surface portions 617 of the pad body face outward away from one another and the curved outer surface portions 621 face toward one another. As thus configured, the pad is placed, hinge first, between two toes which require spacing for treatment (see FIGS. 48 and 49). Placement of the pad between the toes will cause the curved faces of the outer surface portions 621 to resiliently contact one another and provide a spring force tending to separate the toes. It will be noted in this regard that while the material out of which the pad body 601 is made is generally a pliable material, the material is sufficiently resilient when compressed to provide a small spring force. The teardrop shape helps provide the spring force, and allows the spring force to be weaker near the hinge 609, and stronger on the other end, creating a more natural separation of the toes.

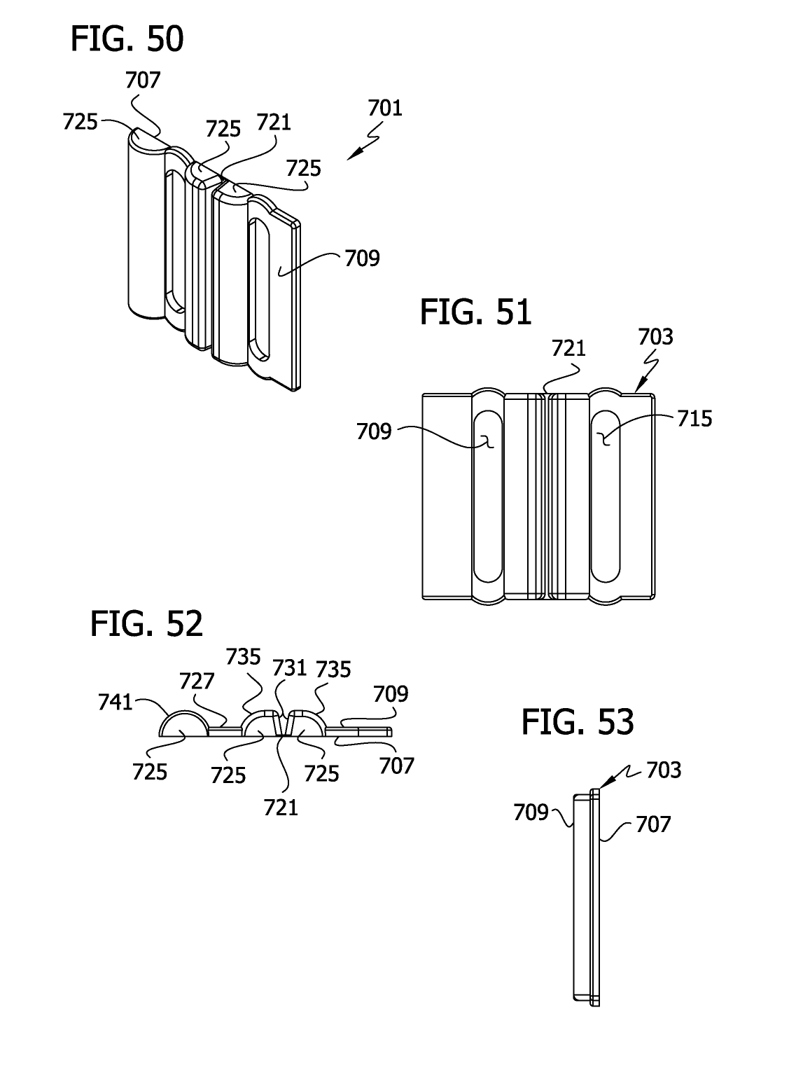

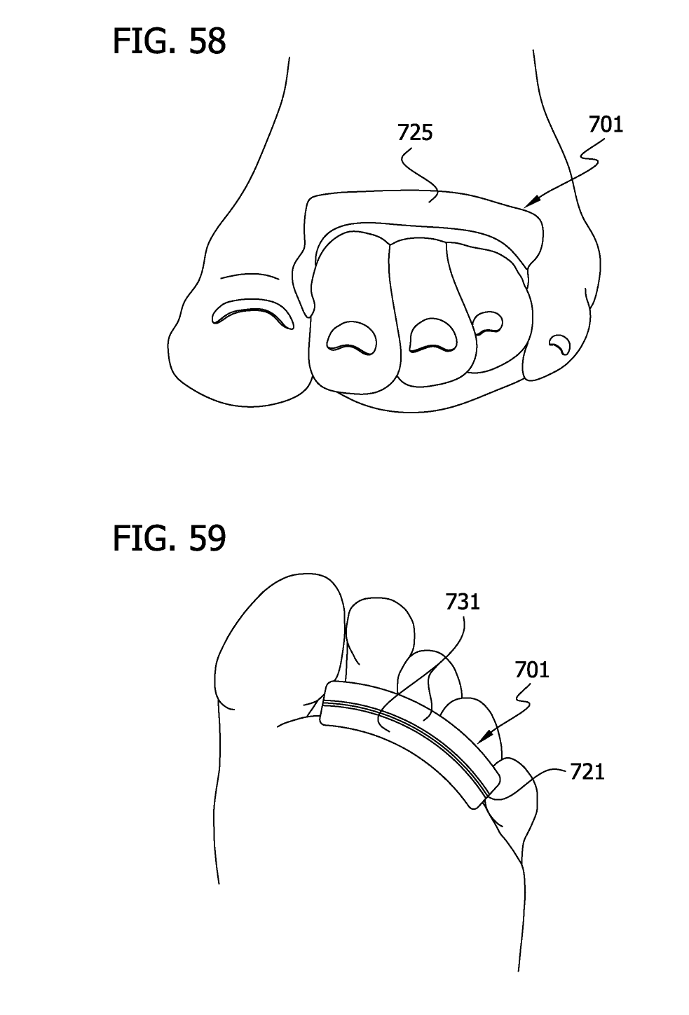

FIGS. 50-57 illustrate one embodiment of a toe crest pad, generally designated 701, for application to the foot as shown in FIGS. 58 and 59. This pad is constructed of the same materials as the all-purpose pad 1 described above. The pad 701 also has an adhesive and backing layer on its inner surface 707 and an antibacterial coating on its outer surface 709, as previously described. However, unlike the previous pads, the pad 701 does not have a recess and thin wall. The pad comprises a generally rectangular pad body 703 having a first opening 709 in it generally adjacent the proximal end of the body and a second opening 715 spaced from the first opening along the longitudinal axis of the body toward the distal end of the body. (As used herein, “proximal” means in the direction toward the head and “distal” means in a direction away from the head.) The openings 709, 715 extend generally transverse to the longitudinal axis of the body and are sized for receiving three toes. A line of weakness forming a hinge 721 extends transverse to the longitudinal axis of the body at a location between the two openings, e.g., about midway between the two openings (see FIGS. 50-52). The pad further comprises a plurality of raised anti-flex portions or ribs 725 extending transverse of the longitudinal axis of the pad from one side of the pad to the other at spaced intervals along the pad. In the illustrated embodiment, three such ribs 725 are shown, first and second ribs on opposite sides of the hinge 721 immediately adjacent the hinge, and a third rib 725 between the distal end of the pad body and the distal opening 715, generally immediately adjacent the distal opening. As illustrated in FIG. 52, the inner surface 707 of the pad body is generally planar. The outer surface 709 of the pad body is generally planar at locations 727 adjacent the ribs. The first and second ribs 725 are generally quadrantal in transverse cross section, having opposing, closely spaced, generally flat faces 731 extending outward from the hinge and outer curved surfaces 735 connecting the flat faces and the planar portions 727 of the outer surface of the pad (FIG. 52). The third rib 725 is generally semi-circular in transverse cross section, having a semi-circular outer curved surface 741.

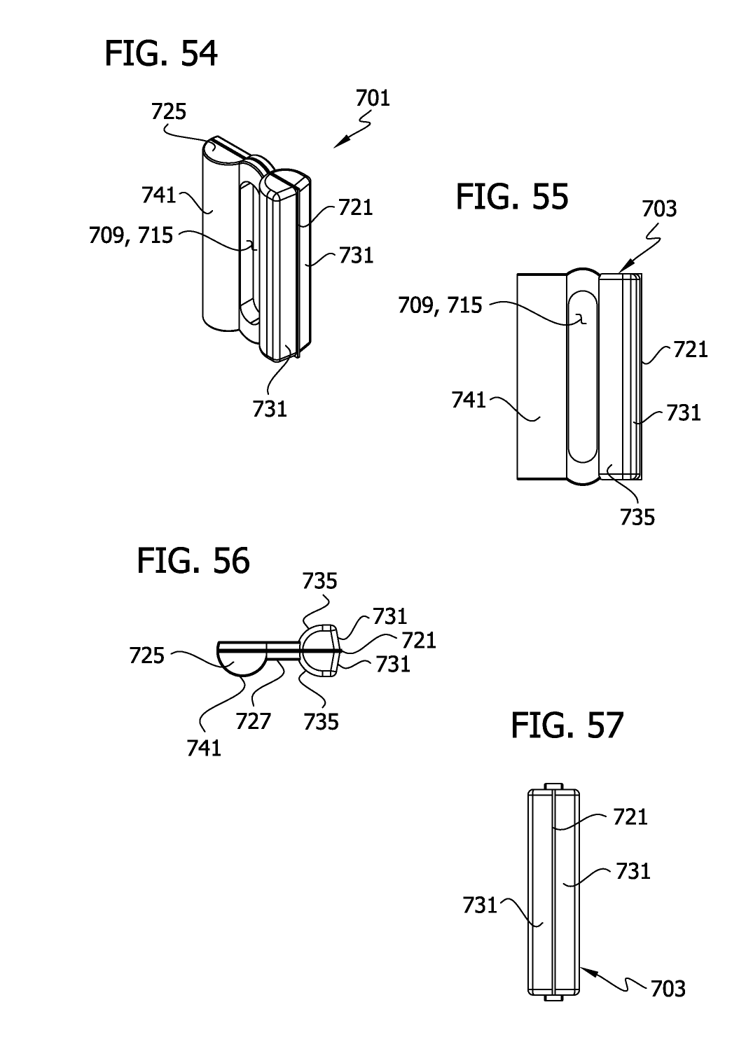

In use the pad body 703 is folded about the hinge 721 to the position shown in FIGS. 54-57 in which the two toe openings 709, 715 are aligned with one another. The pad body 703 is then placed on the foot with the second, third and fourth toes of a foot received in the aligned openings, with the first and second ribs 721 positioned below the three toes generally at the base of the proximal phalanges plantarly, and with the third rib 721 positioned on top of the three toes generally at the metatarsal phalangeal joint (see FIGS. 58 and 59). In this position, the flat faces 731 of the first and second ribs 725 combine to provide a substantially flat surface for contact with portions of footwear underlying the foot, and the outer surface 741 the third rib provides a curved surface for contact with portions of footwear overlying the foot. This arrangement is advantageous over conventional toe crest pads because of the biomechanical design providing elevation of the metatarsal joint axis and allowing the plantar flexors of the toe and extensors of the joints to assist straightening of the metatarsal phalangeal joints.

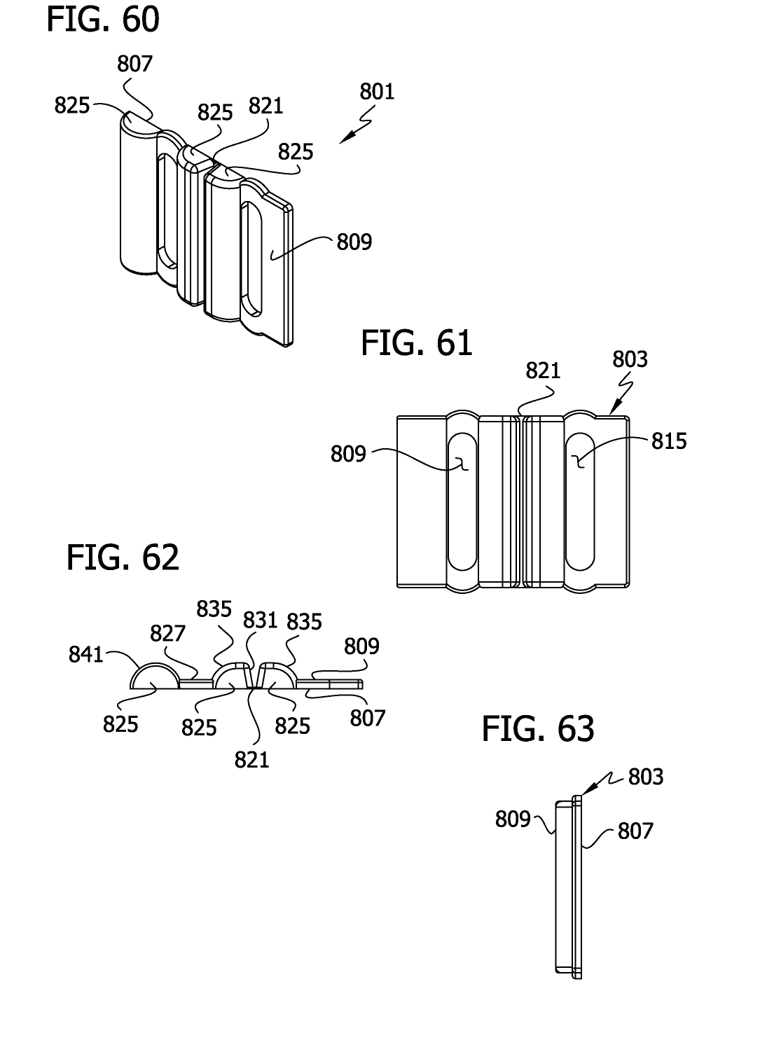

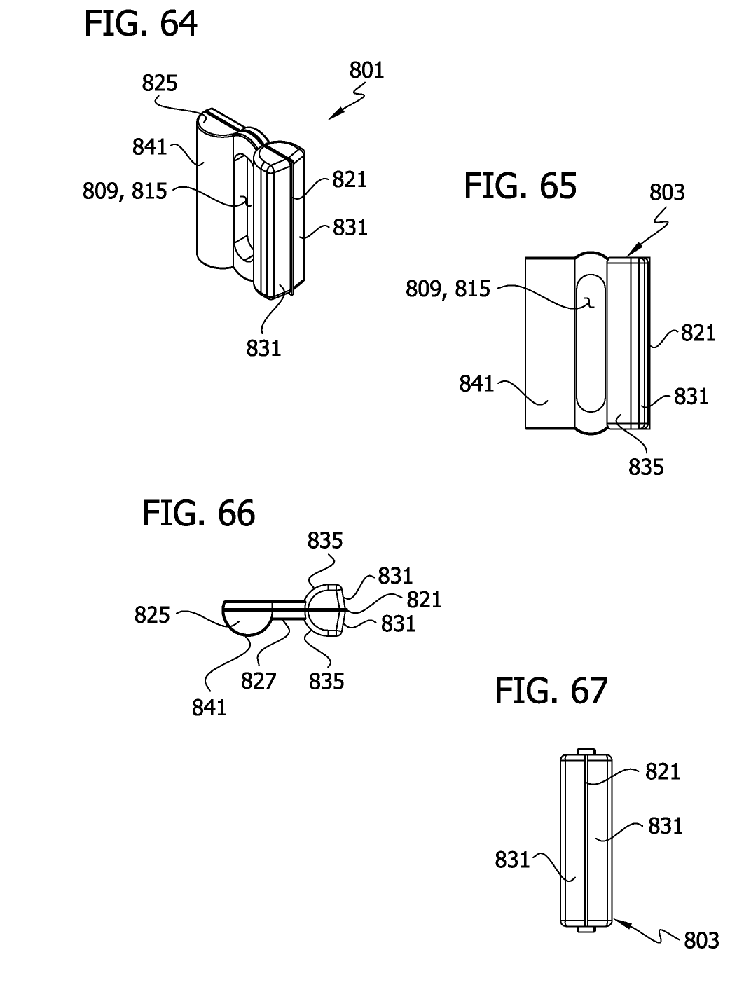

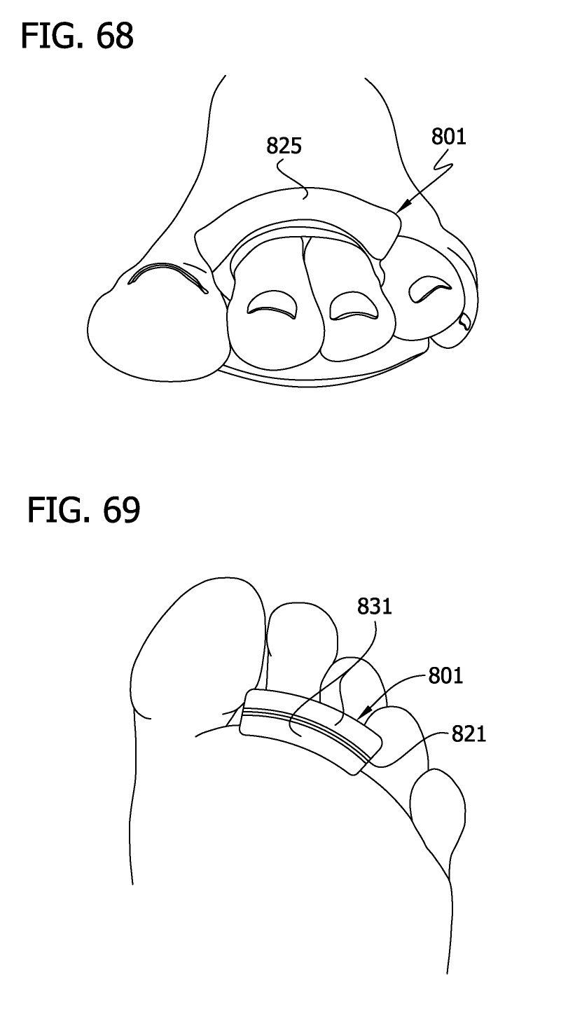

FIGS. 60-67 illustrate a second embodiment of a toe crest pad, generally designated 801, for application to the foot as shown in FIGS. 68 and 69. This pad is constructed in a manner identical to the toe crest pad 701 of the previous embodiment except that the pad is sized to fit two toes instead of three toes.

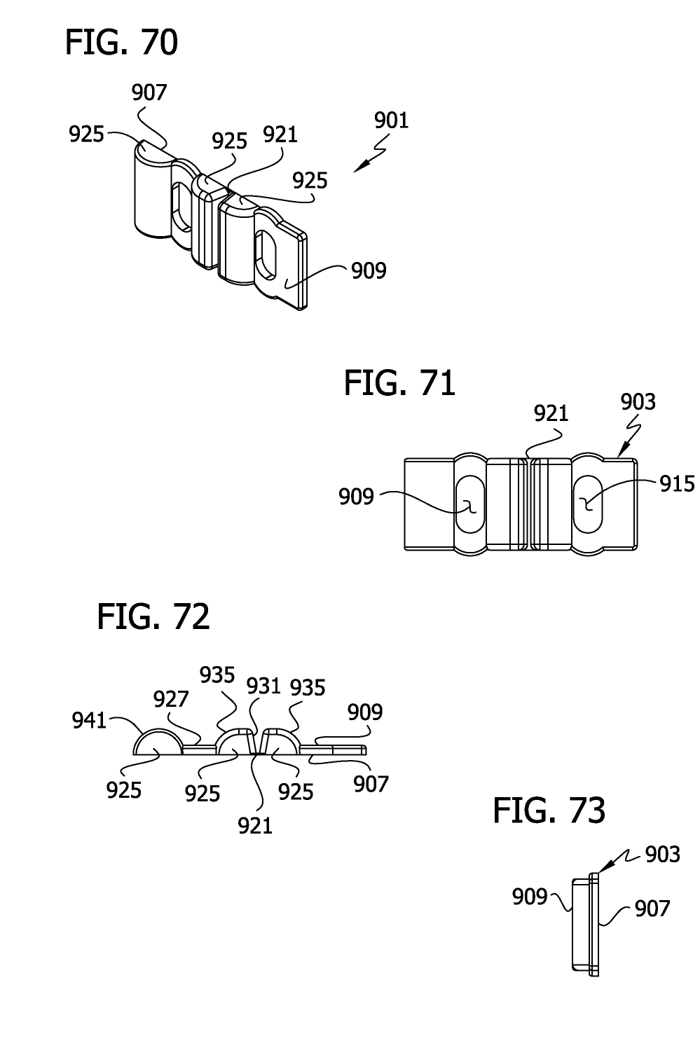

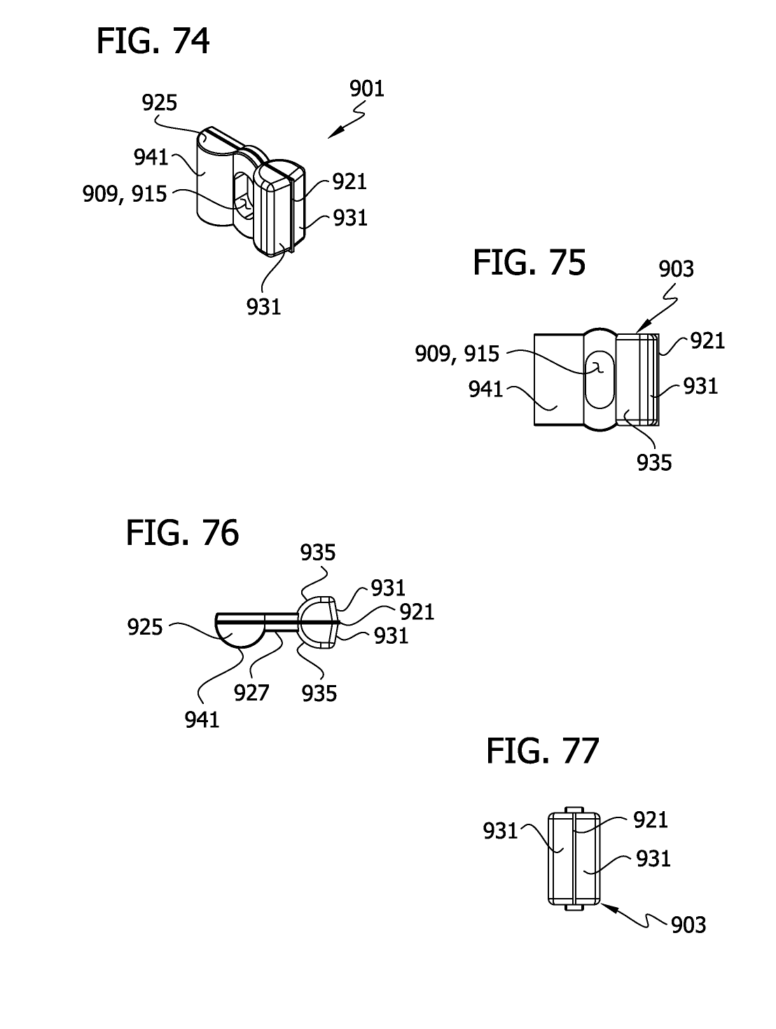

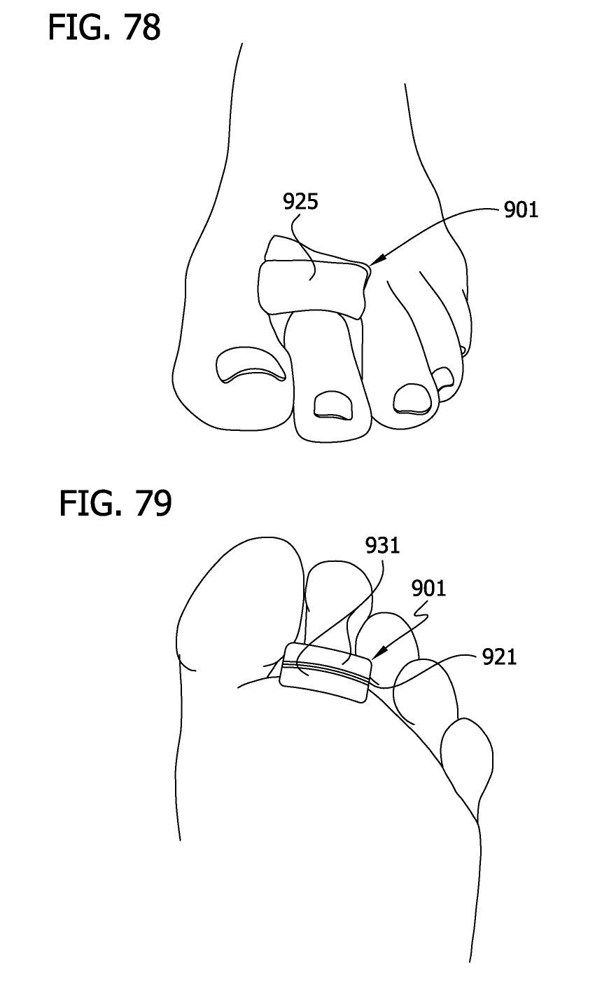

FIGS. 70-77 illustrate a third embodiment of a toe crest pad, generally designated 901, for application to the foot as shown in FIGS. 78 and 79. This pad is constructed in a manner identical to the toe crest pads 701, 801 of the previous embodiments except that the pad is sized to fit one toe instead of two or three toes.

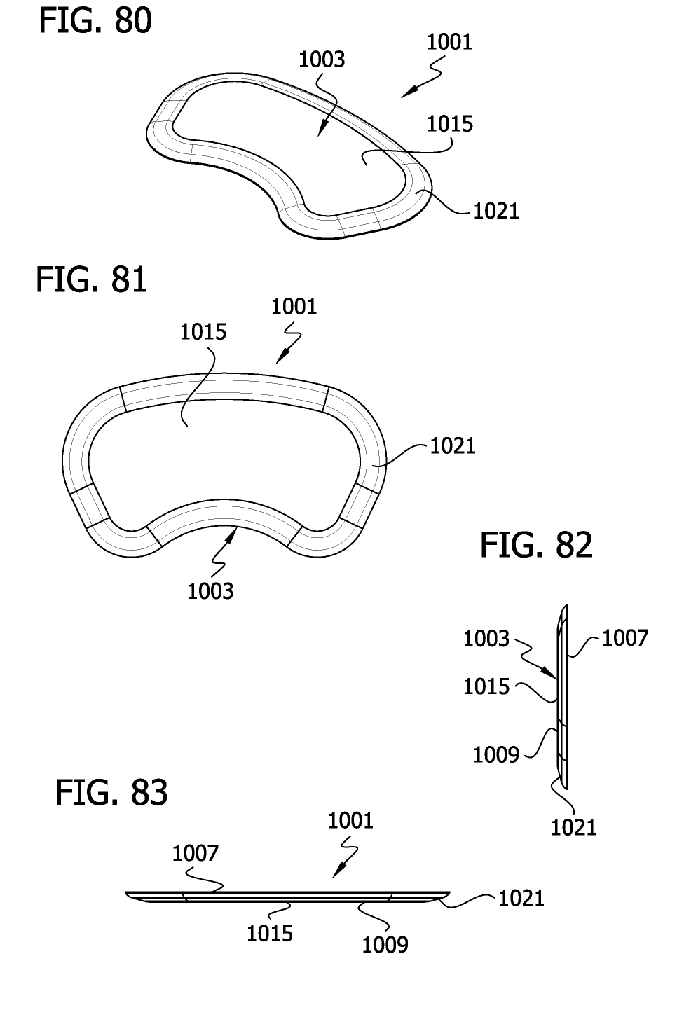

FIGS. 80-83 illustrate one embodiment of a pan metatarsal pad, generally designated 1001, for application to the foot. This pad comprises a pad body 1003 of cushioning material having an inner surface 1007 for contact with the skin and an outer surface 1009. By way of example, the cushioning material may be a soft, pliable, gel-like material (e.g., polyurethane) which is sufficiently pliable and flexible to mold itself to the contours of the body when applied to the skin. In one embodiment, the pad body has a Shore OO hardness of about 45-70 (+/−7). The material is desirably substantially transparent (so that it is not readily visible on the skin), non-toxic when applied to the skin, hypoallergenic and water resistant. One suitable polyurethane material for use in making the pad body is commercially available from PolyWorks Inc. (24 Albion Road, Suite 101, Lincoln, RI 02865.

The pad body 1003 has a central area 1015 of substantially uniform thickness and a periphery 1021 which is skived to provide greater area for adhesion to the foot. By way of example but not limitation, the central area 1015 of the pad body may have a thickness in the range of 0.075 in. to about 0.15 in. (e.g., about 0.10 in. and about 0.135 in.). The pad 1201 also has an adhesive and backing layer on its inner surface 1205 and an antibacterial coating on its outer surface 1207, as previously described. The purpose of the pad 1001 is to evenly balance the weight applied to all five metatarsals using the properties of polyurethane. This pad, unlike metatarsal pads which are applied to individual metatarsals or to two or three metatarsals (maximally), is sized to extend from the medial to the lateral side of the foot plantarly, thus protecting all of the metatarsals and providing comfort to individuals having decreased fat pads through genetics, diseases and/or aging.

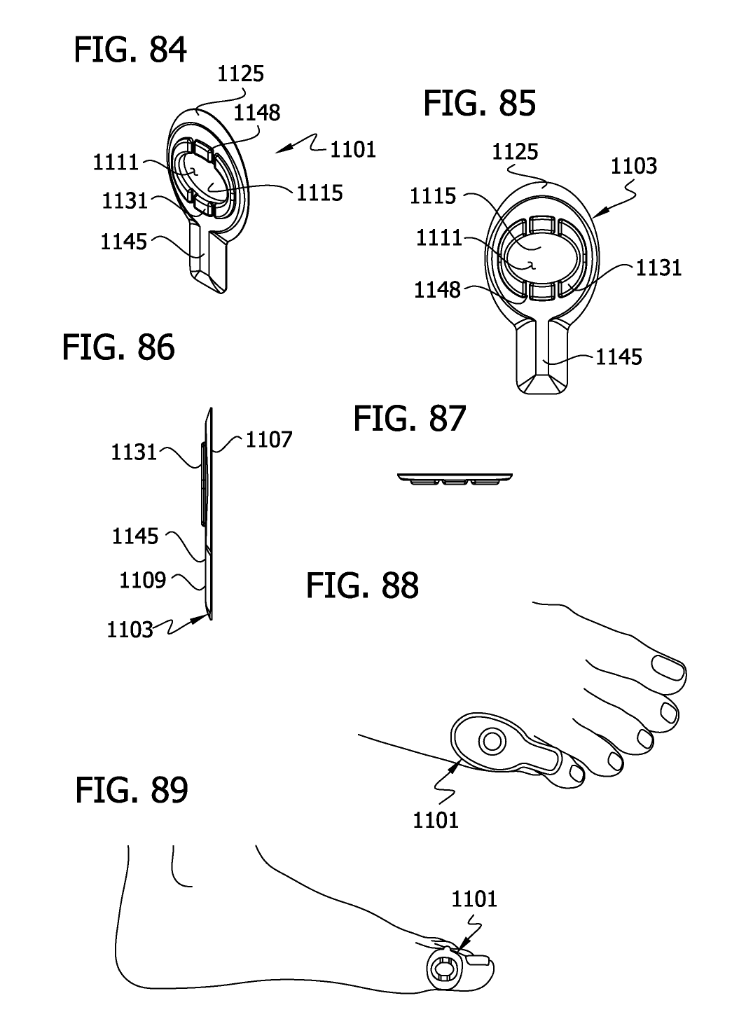

FIGS. 84-87 illustrate one embodiment of a pinch callous pad, generally designated 1101, for application to the foot as shown in FIGS. 88 and 89. This pad is constructed in much the same manner as the all-purpose pad 1 described above, and corresponding parts are indicated by corresponding reference numbers plus 1100. The pad body 1103 has a recess 1111 and thin wall 1115 located generally centrally of the pad. The pad also has an adhesive and backing layer on its inner surface 1107 and an antibacterial coating on its outer surface 1109, as previously described. The peripheral margin 1125 of the pad comprises a skived edge leading to an elongated finger 1145 for adhesively securing the pad body 1103 to the skin. In use, the pad body 1103 is positioned on the skin with the recess 1111 and thin wall 1115 aligned with a callous on a foot (see FIGS. 88 and 89). If medication is used, a quantity of the medication is received in the cavity created by the deflection of the thin wall 1115 into the recess 1111. As in the previous pads 1, 101, 201, 301, and 401, the pad has a pressure relief ridge 1131, which in this embodiment is split into sections 1148 to allow it to wrap more readily around small curved surfaces of the foot.

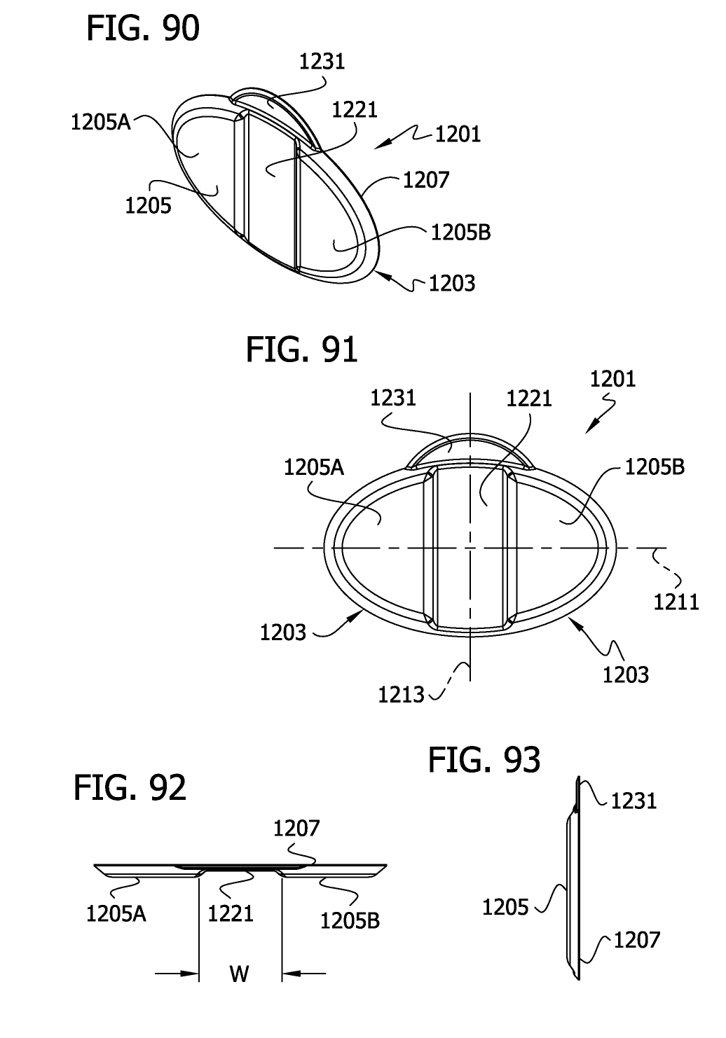

FIGS. 90-93 illustrate one embodiment of so-called “bi-plane” pad, generally designated 1201, comprising a pad body 1203 of cushioning material having an inner surface 1205 adapted to contact the skin and an outer surface 1207. By way of example, the cushioning material may be a soft, pliable, gel-like material (e.g., polyurethane) which is sufficiently pliable and flexible to mold itself to the contours of the body when applied to the skin. In one embodiment, the pad body has a Shore OO hardness of about 45-70 (+/−7). The material is desirably substantially transparent (so that it is not readily visible on the skin), non-toxic when applied to the skin, hypoallergenic and water resistant. One suitable polyurethane material for use in making the pad body is commercially available from PolyWorks Inc. (24 Albion Road, Suite 101, Lincoln, RI 02865. The pad 1201 also has an adhesive and backing layer on its inner surface 1205 and an antibacterial coating on its outer surface 1207, as previously described.

In the embodiment of FIGS. 90-93, the pad body 1203 is generally oval-shaped and has a central longitudinal axis 1211 extending lengthwise of the pad and a central transverse axis 1213 extending widthwise of the pad. Further, the inner surface 1205 is recessed to have a recess or groove 1221 extending along the central transverse axis 1213 across the pad body 1203. The groove 1221 is generally of channel shape with a substantially flat bottom and inclined (outwardly divergent) side walls extending up from the bottom. The inner surface 1205 has side regions 1205A and 1205B on opposite sides of the recess 1221. When the pad is in a relaxed condition off the foot, these side regions 1205A, 1205B are generally co-planar. An adhesive tab 1231 projects from one side of the pad body 1203 for adhesively securing the pad to the body and also stabilizing the pad. In this embodiment, the tab 1231 is generally aligned with the groove 1221 and has an inner surface generally co-planar with the bottom of the groove and an outer surface generally co-planar with the outer surface 1207 of the pad body. The recess 1221 has a width W (in the direction of the longitudinal axis 1211) sufficient to relieve pressure on specific portions of a foot to which the pad is applied, as discussed below. The pad body 1203 has a thickness at opposite sides of the recess 1221 in the range of about 0.1 in to about 0.2 in. (e.g., about 0.12 in. and about 0.19 in.).

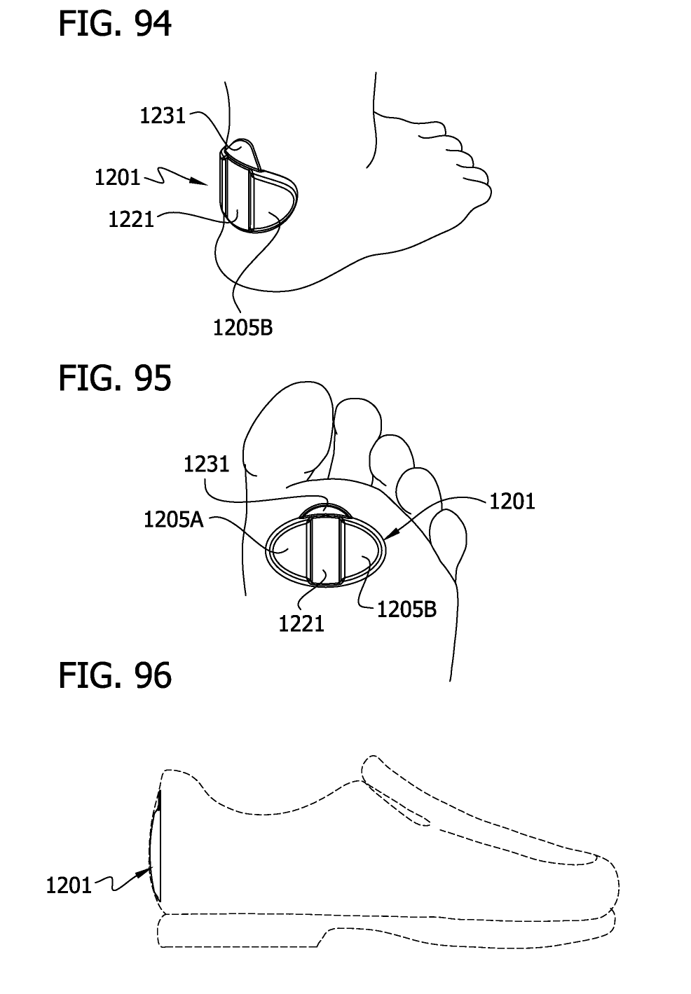

In use, the pad 1201 can be positioned on the skin at different locations, depending on the treatment. FIGS. 94-96 show several different applications. In particular, FIG. 94 shows the pad 1201 positioned on the bottom of the foot for reducing pressure on the second, third and fourth metatarsal heads by suspending them over the recess 1221, the first and fifth metatarsal heads being supported by the side regions 1205A, 1205B of the pad body. For this application, the width W of the recess is preferably in the range of about 0.6 in. to about 1.0 in., and the depth of the recess is preferably in the range of about 0.062 in. to about 0.187 in. Essentially the same design can be used to reduce the pressure on one or two metatarsal heads with a corresponding reduction in the width W of the recess.

FIG. 95 shows the pad 1201 positioned on the heel of the foot with the vertical boney prominence on the back of the heel received in the vertically-oriented recess 1221 to relieve pressure at that location. FIG. 96 shows the pad secured (e.g., by adhesive) in footwear, e.g., a shoe, with the recess 1221 in a generally vertical orientation and positioned for receiving the vertical boney prominence on the back of the heel of a foot received in the shoe to relieve pressure at that location.

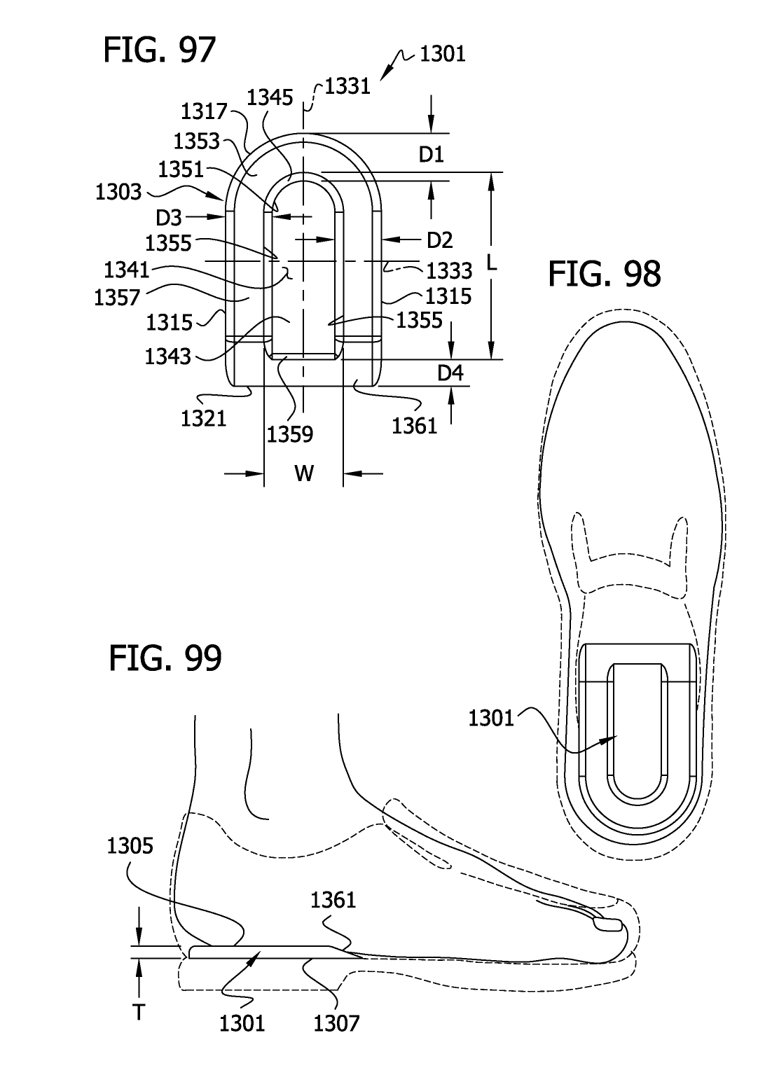

FIGS. 97-99 illustrate one embodiment of a heel lift of the invention, generally designated by the reference number 1301. The heel lift 1301 comprises a lift body 1303 of cushioning material having an inner surface 1305 adapted to contact the skin of a foot and an outer surface 1307 adapted to contact footwear in which the foot is received. (In this context, “footwear” includes shoes, boots, sandals, insoles, orthotics and related devices.) By way of example, the cushioning material may be a soft, pliable, gel-like material (e.g., polyurethane) which is sufficiently pliable and flexible to mold itself to the contours of the wearer when applied to the skin. In one embodiment, the lift body 1303 has a Shore OO hardness of about 45-70 (+/−7). The material is desirably substantially transparent (so that it is not readily visible on the skin), non-toxic when applied to the skin, hypoallergenic and water resistant. One suitable polyurethane material for use in making the pad body is commercially available from PolyWorks Inc. (24 Albion Road, Suite 101, Lincoln, RI 02865. The lift 1301 also has an adhesive and backing layer on its inner surface 1305 and an antibacterial coating on its outer surface 1307, as previously described.

The heel lift body 1303 has a generally U-shaped outline comprising substantially straight side edges 1315, a rounded back edge 1317, and a substantially straight front edge 1321. The heel lift body has a central longitudinal axis 1331 extending lengthwise of the lift body and a central transverse axis 1333 extending widthwise of the body. Further, the inner surface 1305 is recessed to have a recess or depression 1341 which extends a major portion of the length and width of the lift body 1303. The recess has a substantially flat bottom 1343 and inclined side walls 1345. Further, in this embodiment, the recess 1341 has a shape generally corresponding to the generally U-shaped outline of the overall lift body 1303. In particular, the recess has a rounded back edge 1351 spaced a distance D1 from the back edge 1317 of the lift body (defining a rear region 1353 of the inner surface of the lift body), substantially straight side edges 1355 spaced distances D2 and D3 from respective side edges of the body 1303 (defining side regions 1357 of the inner surface of the lift body), and a front edge 1359 spaced a distance D4 from the front edge of the body (defining a front region 1361 of the inner surface of the lift body). The recess 1341 has an overall length L from its back edge 1351 to its front edge 1359 and an overall width W between its side edges 1355. The recess also has an overall depth of about 0.1 in. These dimensions will vary depending on the size of the foot, but the recess should be sized to comfortably receive the bottom central portion of the heel. For example, the dimensions of the recess 1341 may be as follows for an adult male:

Size (Adult Male)

D1—0.670 in.

D2—0.650 in.

D3—0.650 in.

D4—0.375 in.

L—2.629 in.

W—0.881 in.

Recess depth—0.09 in.

The above dimensions may vary.

The thickness T of the lift body 1303 will vary depending on the desired amount of lift to be provided to the foot. Exemplary thicknesses are in the range of about 0.1 in. to about 0.4 in. (e.g., about 0.125 in., about 0.25 in. and about 0.375 in.). The front region 1361 of the inner surface of the heel lift tapers down to the front edge 1321, which is relatively thin (e.g., about 0.01 in.) for comfort.

FIGS. 98 and 99 illustrate placement of the heel lift 1301 in a shoe for supporting a heel of a person wearing the shoe. The central portion of the heel is received in the recess 1341 to relieve pressure on the heel. Opposite side portions of the heel are supported by the side regions 1357 of the heel lift on opposite sides of the recess. The length of the lift is such that it extends from a location closely adjacent the back of heel to a location at which the front region 1361 of the inner surface of the lift body extends under the arch of the foot.

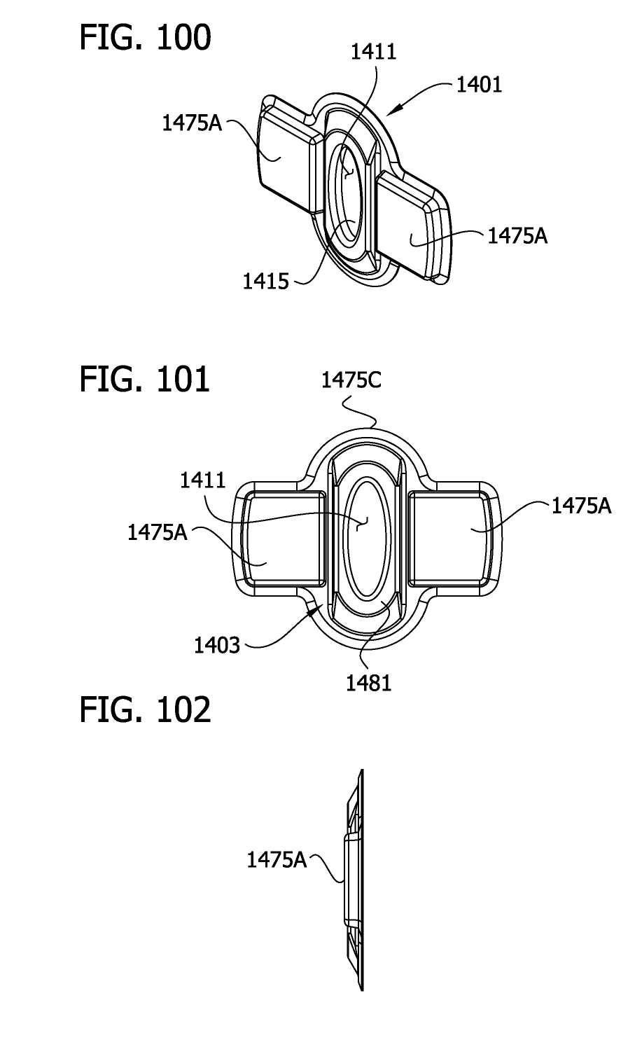

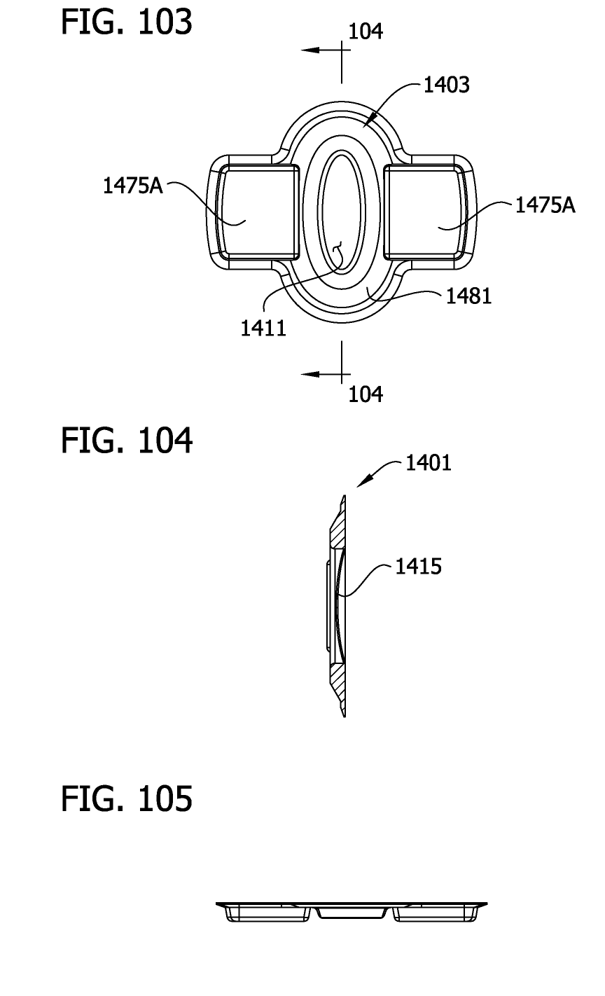

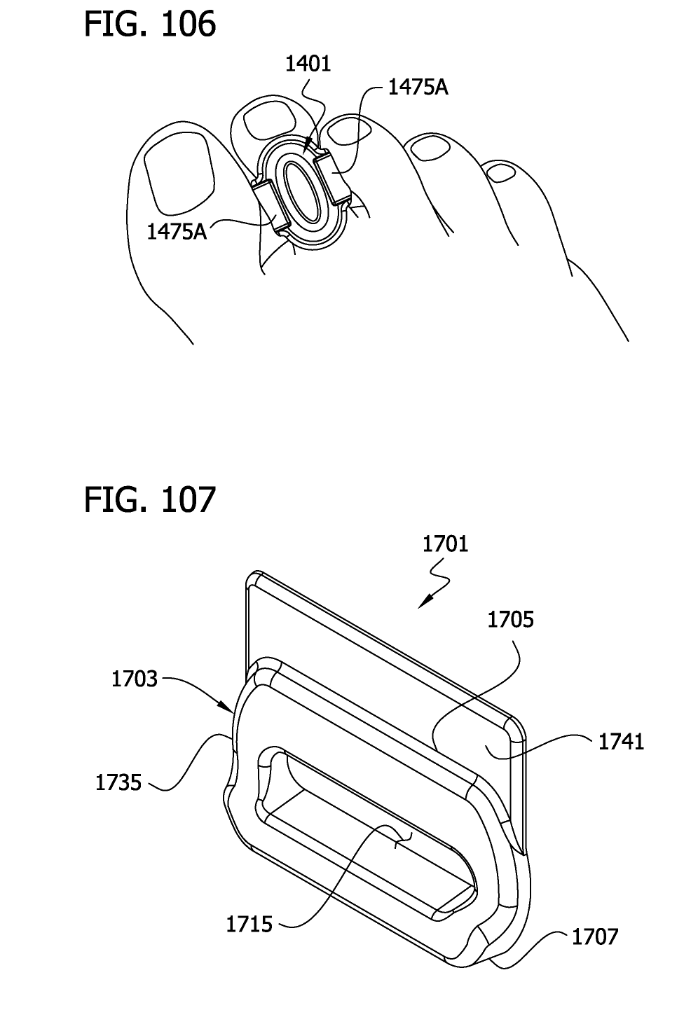

FIGS. 100-105 illustrate an embodiment of a corn hammertoe pad, generally designated 1401, for application to the foot as shown in FIG. 106. This pad 1401 is constructed in much the same manner as the corn pad 401 described above, and corresponding parts are indicated by corresponding reference numbers plus 1000. The pad has a recess 1411 and thin wall 1415 located generally centrally of the pad body 1403. The pad also has an adhesive and backing layer on its inner surface and an antibacterial coating on its outer surface, as previously described in connection with pads 1 and 401. The peripheral margin of the pad comprises a plurality of securing flaps 1475 for adhesively securing the pad body 1403 to the skin. More specifically, in one embodiment, four such flaps are provided, two relatively large lateral flaps 1475A at opposite sides of the pad body 1403, a proximal flap 1475B at the proximal end of the pad body, and a distal flap 1475C at the distal end of the pad body. The two lateral flaps 1475A are illustrated as being generally rectangular and sufficiently thick (e.g., in the range of about 0.1 in. to about 0.15 in., such as about 0.12 in.) so that they can effectively function as toe spacers to maintain the toes spaced apart when the central portion of the pad 1401, having a thickness of about 0.1 in., is applied to the top of a toe with the lateral flaps extending down on opposite sides of the toe (FIG. 106). The proximal and distal flaps 1475B, 1475C are rounded in this embodiment, but they may have other shapes. As in the previous pads, e.g., 1, 101, 201, 301, 401, the pad 1401 has a pressure relief ridge 1481 extending around the recess 1411 on the outer surface of the pad.

In use, the pad body is positioned with its inner surface in contact with the skin and the recess 1411 and thin wall 1415 aligned with a corn area on top of a toe of the foot (see FIG. 106). (This area generally corresponds to the area of the proximal interphalangeal joint or distal interphalangeal joint.) If medication has been applied to the corn area, the thin wall 1415 of the pad will deflect outward into the recess 1411 to form a cavity to contain the medication (see FIG. 104), as previously described in earlier embodiments.

When the pad body 1403 is properly positioned on the top of the toe, the lateral flaps 1475A are extended down on opposite sides of the toe. The flaps function to maintain the two adjacent toes at an appropriate spacing from the middle toe. Also, the lateral flaps protect the skin against irritation from bony protuberances on the toes and help avoid the formation of corns.

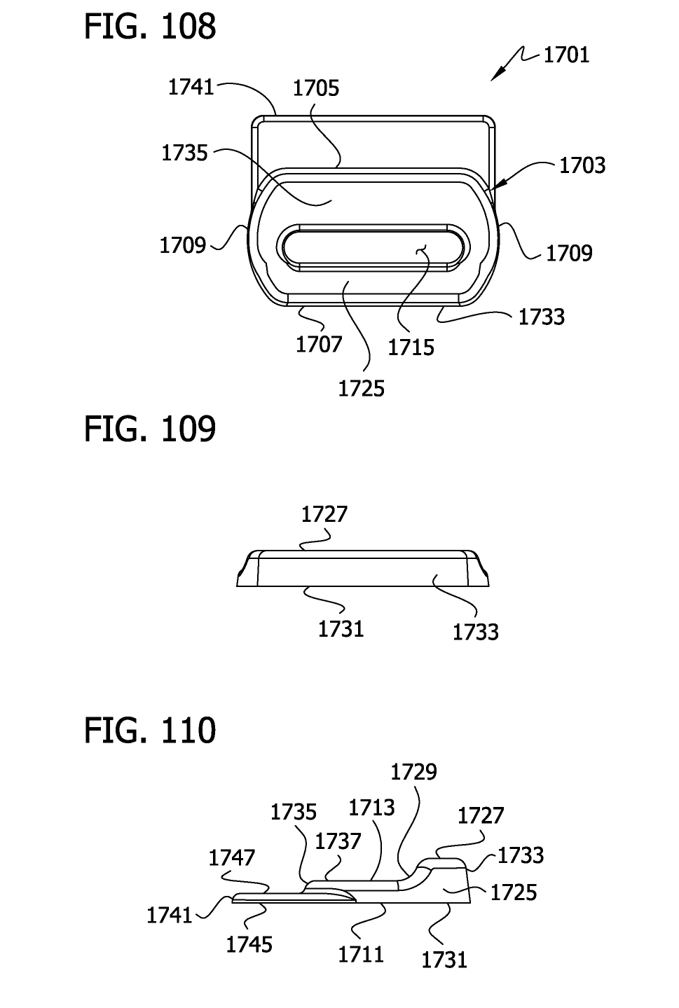

FIGS. 107-111 illustrate a fourth embodiment of a toe crest pad, generally designated 1701, for application to the foot as shown in FIGS. 112-114. This pad is constructed of the same materials as the all-purpose pad 1 and the toe crest pads 701, 801 and 901 described above. The pad 1701 comprises a pad body 1703 having a proximal end 1705, a distal end 1707, opposite rounded sides 1709, an inner surface 1711 and an outer surface 1713. The pad has an adhesive and backing layer on its inner surface 1711 and an antibacterial coating on its outer surface 1713, as previously described. An elongate opening 1715 extends lengthwise of the body between opposite sides of the body and is sized for receiving a number of toes. In the embodiment of FIGS. 107-110, the opening 1715 is sized for three toes, but this number may vary. The distal end margin of the pad body 1703 is relatively thick (e.g., in the range of about 0.3 in. to about 0.4 in., such as about 0.375 in.) to form a distal anti-flex portion or first rib 1725 extending from adjacent one side of the pad body to adjacent the opposite side of the pad body. As best illustrated in FIG. 110, the rib 1725 has a generally planar top face 1727, a concave proximal face 1729, a generally planar bottom face 1731 extending substantially parallel to the top face 1727, and a generally planar distal end face 1733. The proximal end margin of the pad body 1703 is sufficiently thick (e.g., in the range of about 0.2 in. and about 0.35 in., such as about 0.30 in.) to provide a proximal anti-flex portion or second rib 1735 extending from adjacent one side of the pad body to adjacent the opposite side of the pad body. As viewed in FIG. 110, the proximal anti-flex portion 1735 has a top face 1737 offset below the top face 1727 of the distal anti-flex portion 1725. A rectangular pull tab 1741 extends in a proximal direction from the proximal end 1741 of the pad body 1703 for assisting in the application of the pad to the foot and for providing additional surface area for adhesion of the pad to the foot. When the pad is in its relaxed condition (e.g., FIG. 110), the inner surface 1711 of the pad body 1703 is desirably generally planar and generally co-planar with the inner surface 1745 of the pull tab. The pull tab 1741 has a generally planar outer surface 1747 offset below the top face 1737 of the proximal anti-flex portion 1735 of the pad.

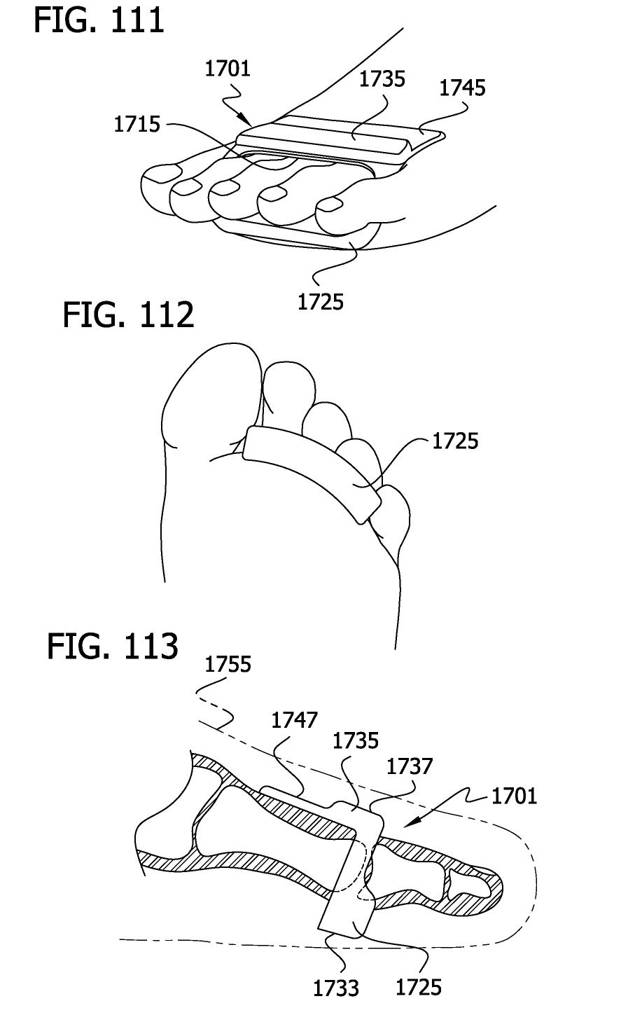

In use, the pad body 1703 is placed on the foot as shown in FIGS. 111-113, that is, with the second, third and fourth toes of a foot received in the opening 1715. The pull tab 1741 is then pulled to position the pad so that the distal anti-flex portion 1725 is below the three toes generally at the base of the proximal phalangeal joints (MPJ's), and the proximal anti-flex portion 1735 is on top of the foot generally at the metatarsal phalangeal joints of the three toes directly opposite (above) the distal anti-flex portion. The pull tab 1741 is then pressed against the foot to adhere the pad in the desired position in which the proximal face 1729 of the anti-flex distal portion 1725 presses up against the bottom of the toes against the shafts of the proximal phalanges and the proximal and distal anti-flex portion 1735, 1725 apply opposing forces to the metatarsal phalangeal joints of the three toes. The distal end face 1733 of the distal anti-flex portion 1725 provides a substantially flat surface for contact with portions of footwear 1755 (FIG. 113) underlying the foot, and the top faces 1747, 1737 of the pull tab and proximal anti-flex portion 1735 provide substantially flat surfaces for contact with portions of footwear overlying the foot. This arrangement is advantageous over conventional toe crest pads because of the biomechanical design providing elevation of the metatarsal joint axis and allowing the plantar flexors of the toe and extensors of the joints to assist straightening of the metatarsal phalangeal joints.

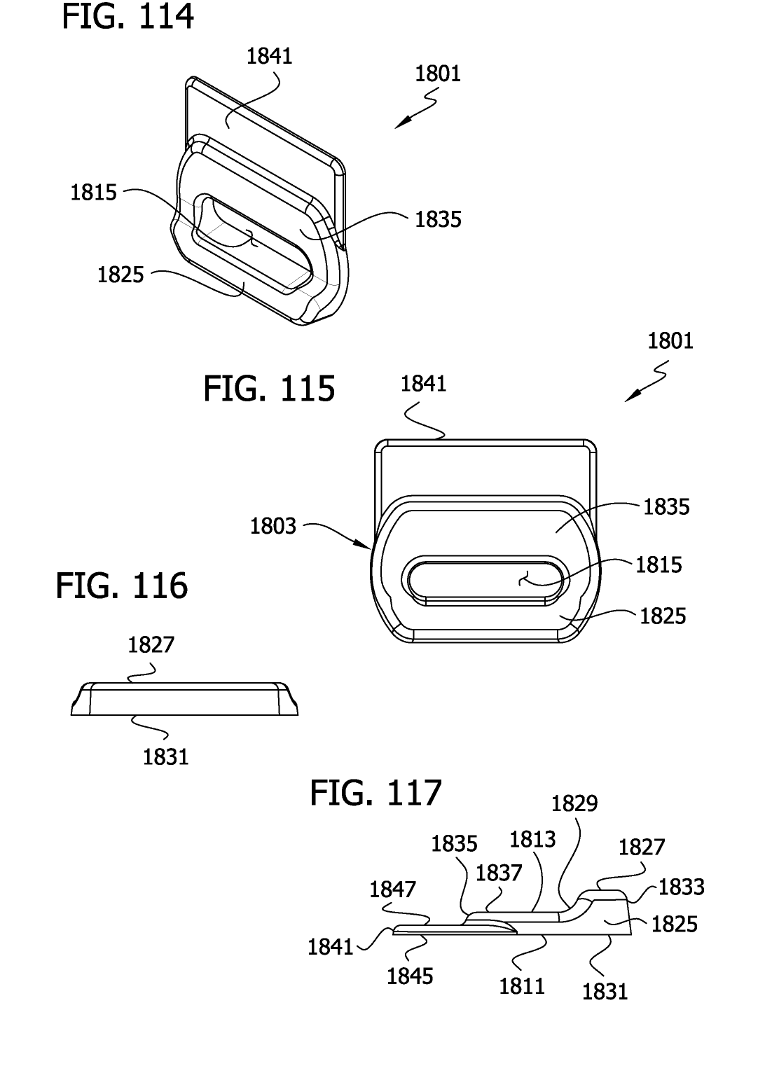

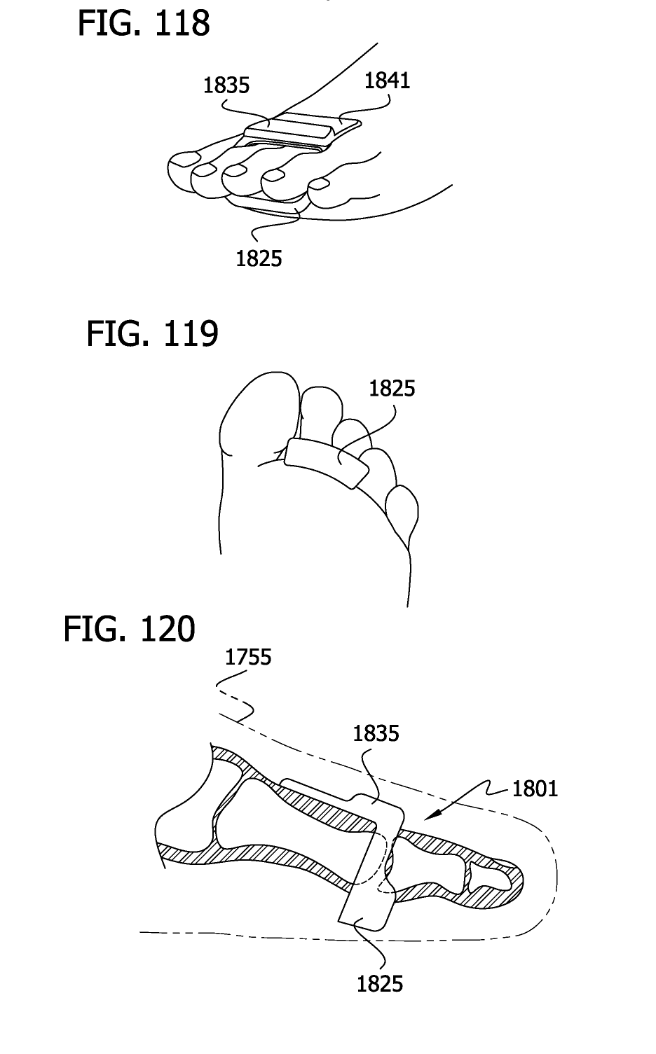

FIGS. 114-117 illustrate a fifth embodiment of a toe crest pad, generally designated 1801, for application to the foot as shown in FIGS. 118-120. This pad is constructed in a manner substantially identical to the toe crest pad 1701 of the previous embodiment and corresponding parts are designated by corresponding reference numbers plus 100. The main difference between the two pads is that the pad 1801 is sized to fit two toes instead of three toes. The thickness dimensions are substantially the same.

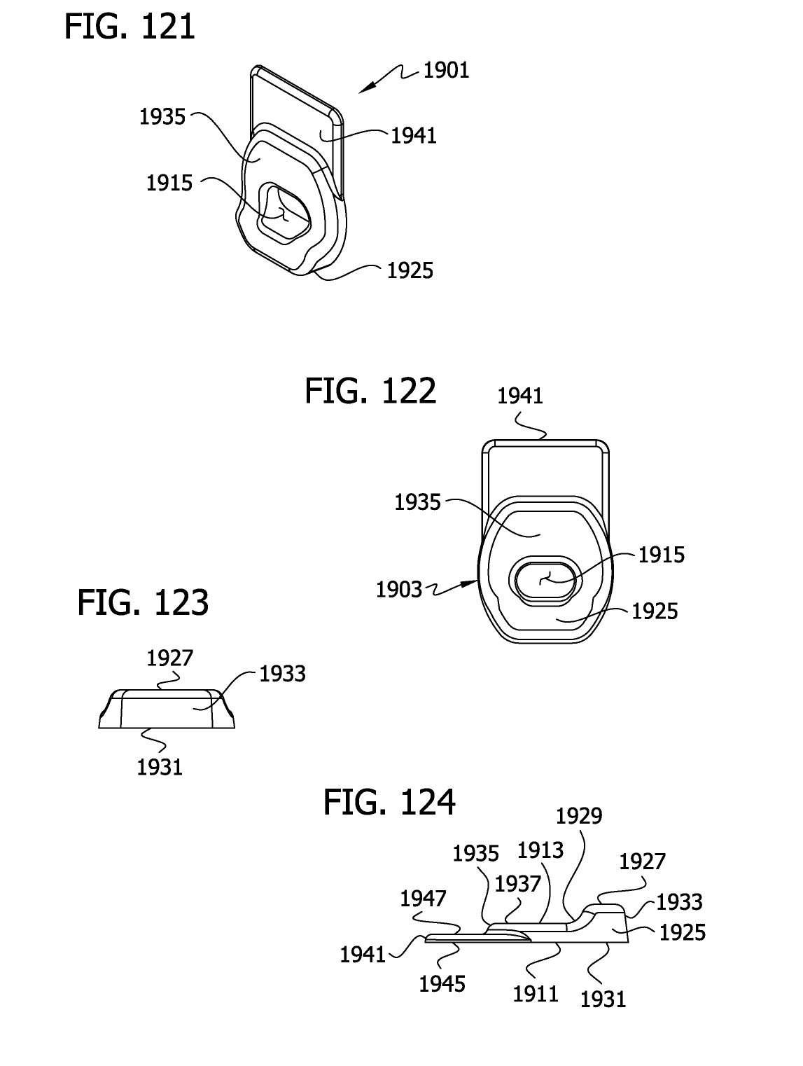

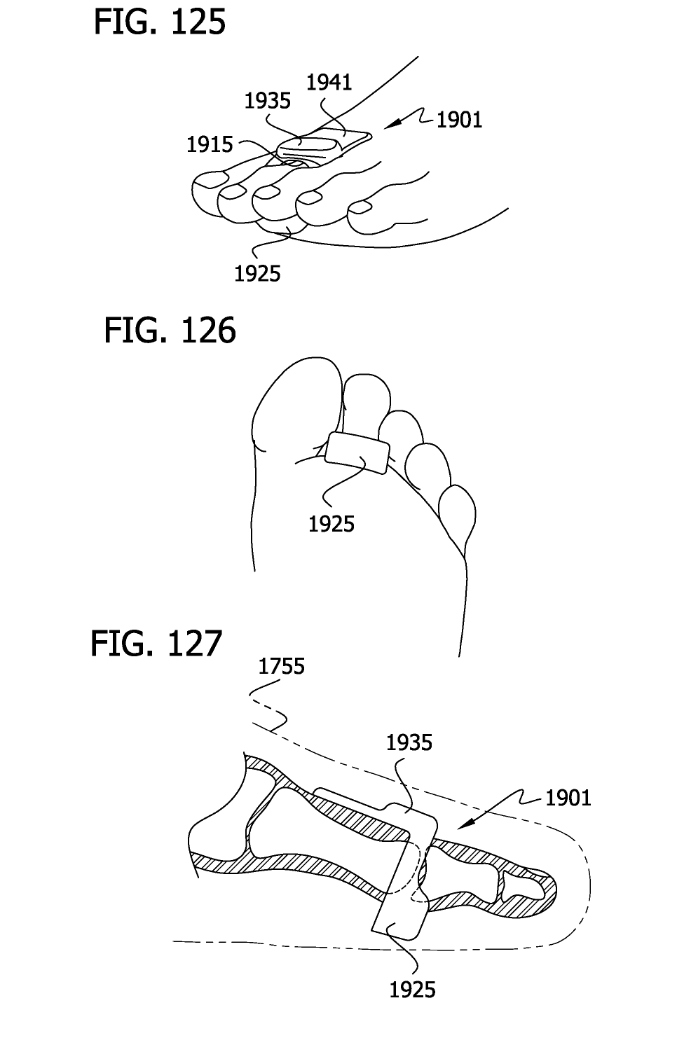

FIGS. 121-124 illustrate a third embodiment of a toe crest pad, generally designated 1901, for application to the foot as shown in FIGS. 125-127. This pad is constructed in a manner substantially identical to the toe crest pad 1701 of the previous embodiment and corresponding parts are designated by corresponding reference numbers plus 200. The main difference between the two pads is that the pad 1901 is sized to fit one toe instead of three toes. The thickness dimensions are substantially the same.

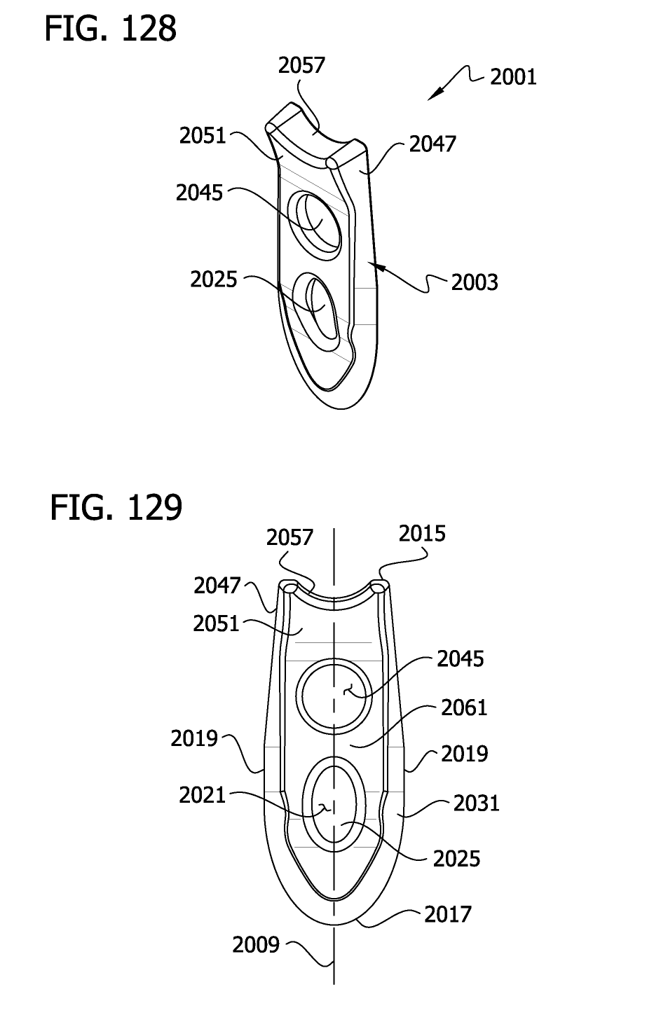

FIGS. 128-131 illustrate one embodiment of a bunion toe spacer pad generally designated 2001. The pad comprises a pad body 2003 of cushioning material having an inner surface 2005 adapted to contact the skin and an outer surface 2007. The material may be of the same type used to make the pad 1. By way of example, the cushioning material may be a soft, pliable, gel-like material (e.g., polyurethane) which is sufficiently pliable and flexible to mold itself to the contours of the body when applied to the skin. In one embodiment, the pad body 2003 has a Shore OO hardness of about 45-70 (+/−7). The material is desirably substantially transparent (so that it is not readily visible on the skin), non-toxic when applied to the skin, hypoallergenic and water resistant. One suitable polyurethane material for use in making the pad body 2003 is commercially available from PolyWorks Inc. (24 Albion Road, Suite 101, Lincoln, RI 02865. The pad 2001 also has an adhesive and backing layer on its inner surface 2005 and an antibacterial coating on its outer surface 2007, as described with respect to previous embodiments.

In the embodiment of FIGS. 128-131, the pad body 2003 is elongate and has a central longitudinal axis 2009 extending lengthwise of the pad. The pad body 2003 has a distal end 2015, a proximal end 2017, and opposite sides 2019. The sides 2019 of the pad body are tapered and rounded toward the proximal end 2017 of the body as viewed in FIG. 129. A recess 2021 is provided generally toward the proximal end 2017 of the pad body. The recess 2021 extends from the outer surface 2007 of the pad body toward the inner surface 2005 of the pad body a distance which is less than the overall thickness of the body. As a result, a thin, flexible wall 2025 is provided at the bottom of the recess 2021, as in some of the previous embodiments (e.g., pad 1). As described earlier in regard to some of such previous embodiments, the wall 2025 is adapted to deform outwardly into the recess 2021 above it to provide a cavity between the pad body 2003 and the skin for holding a small volume of medication which has been pre-applied to the skin (see e.g., cavity 17 in FIG. 4A). Again, by way of example but not limitation, the thin wall 2025 may have a thickness of about 0.010 in., but this dimension may vary. In the particular embodiment of FIGS. 128-131, the thin wall 2025 is formed as an integral part of the pad body, and the bottom surface of the thin wall is generally co-planar with the surrounding portion of the inner surface 2007 of the pad body 2003. However, it will be understood that the thin wall 2025 could be formed as a part separate from the rest of the pad body and then secured in place over the recess 2021. The recess 2021 is generally oval in shape, but this shape may vary. The peripheral margin 2031 of the pad body is skived or tapered from the outer surface 2007 to the inner surface 2005 of the pad body 2003 and provides additional area at the inner surface of the pad body for adhesively securing the pad in place on the skin.

The inner surface 2005 of the pad body 2003 has an adhesive coating for adhesion to the skin. A protective peel-off backing sheet (not shown, but similar to backing layer 27 in FIG. 6A) is provided to shield the adhesive coating until the pad is ready for use, at which time the backing layer is removed to expose the adhesive for application of the pad to the skin. The backing sheet is of a relatively stiff, clear material (e.g., 2.0 mil Mylar film) having a release coating thereon to facilitate ready removal of the pad body from the backing sheet. The outer surface 2007 of the pad body 2003 comprises a thin layer of antibacterial material. By way of example, this layer may be formed by a 0.75 mil-thick polyurethane film impregnated with silver.

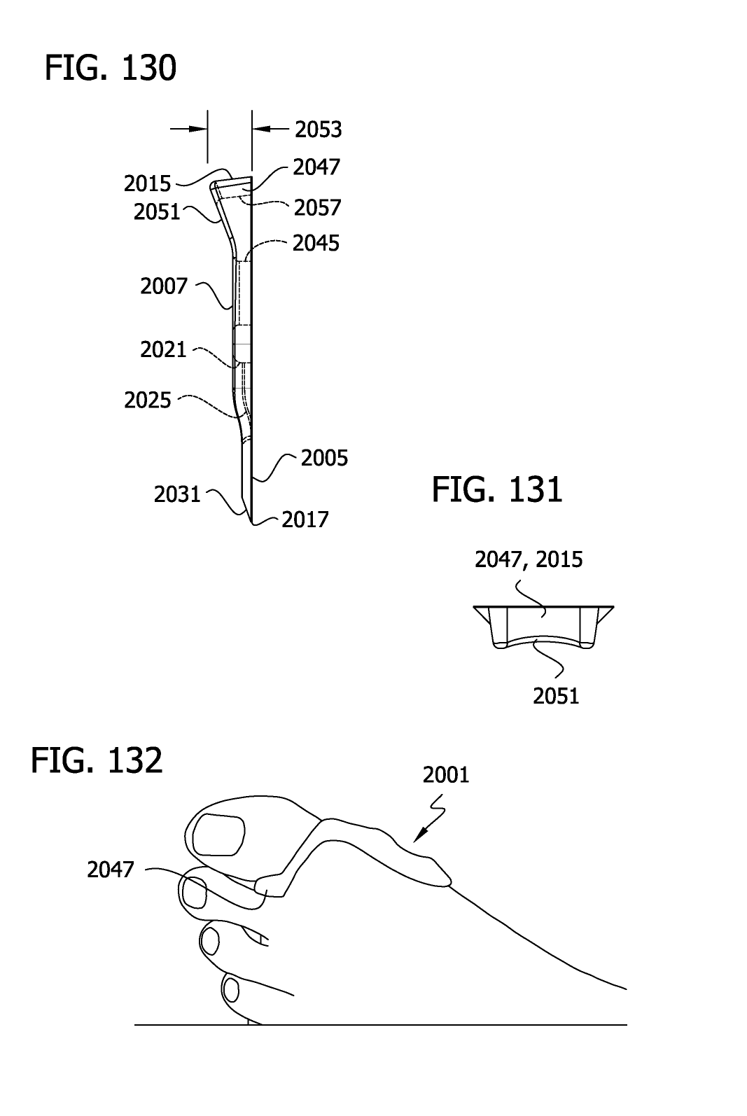

A toe opening 2045 sized for receiving the first toe (the “big” toe) is provided in the pad body 2003 at a location between the recess 2021 and the distal end 2017 of the body. As best illustrated in FIG. 129, a portion of the body pad 2003 between the toe opening 2045 and the distal end 2017 of the pad is configured to be a toe spacer 2047. More specifically, when the pad 2001 is applied to the foot in the manner described below and shown in FIG. 132, the toe spacer 2047 is adapted to be positioned between the first and second toes to maintain a proper separation or gap between the toes. Desirably, the toe spacer 2047 has an outer face 2051 which slopes outward toward the distal end 2015 of the pad from a thickness adjacent the toe opening 2045 of about 0.19 in. to a thickness (indicated at 2053 in FIG. 130) providing the desired gap between the toes. This thickness 2053 is desirably in the range of about 0.375 in. to about 0.425 in., such as about 0.40 in. The edge 2057 defining the distal end 2015 of the pad body 2003 is concave to conform to the interdigital surface (sulcus) of the foot between the first and second toes when the pad is in place.

An exemplary application of the pad 2001 is shown in FIG. 132. The skin is cleaned and dried, and, if needed, a volume of medication is applied to the skin over a bunion area being treated at a location generally corresponding to the first metatarsal phalangeal joint on the medial side of the foot. The backing layer is removed from the pad, and the first toe is inserted through the toe opening 2045 to position the pad as shown in FIG. 132, that is, with the toes spacer 2047 between the first and second toes and the curved distal edge 2057 in contact with the interdigital surface of the foot connecting the first and second toes. The proximal portion of the pad body 2003 is pulled to move the great toe joint to a position appropriate for weight bearing. With the toe in place, the recess 2021 in the pad is aligned with the bunion area and the pad is then pressed against the side of the foot to adhere the pad in position. If medication has been applied to the foot in the bunion area, the thin wall 2025 of the pad body deflects outward into the recess 2021, i.e., toward the outer surface 2007 of the pad body, to create a volume (cavity) for receiving and holding the medication captive at the desired location on the skin. (The deflected position of the wall 2025 is similar to that illustrated in FIG. 4A previously described showing a pad applied to skin over a volume of medication.) On insertion of the foot into footwear, the pressure applied to the pad 2001 by the footwear will be borne by the outer surface 2007 of the pad surrounding the recess 2021, thus relieving the area of the foot below the recess and thin wall from pressure which might otherwise cause pain and/or inhibit treatment of the malady. The portion 2061 of the outer surface 2007 surrounding the recess 2021 and bearing the load on the pad may be referred to as the “pressure ridge” (see FIG. 129).

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above constructions without departing from the scope of the invention, it is intended that all matter contained in the above description and shown in the accompanying drawings shall be interpreted as illustrative and not in a limiting sense. By way of example but not limitation, a pad of this invention could be used on parts of the body other than the foot. For example, a pad could be used on one or more fingers instead of one more toes.

1. A body pad for application to the skin for treating an ailment, said pad comprising
a pad body having an inner surface which is adapted for contact with the skin and an outer surface which faces away from the skin when in use,
a recess in the pad body extending from the outer surface of the pad body toward the inner surface a distance which is less than an overall thickness of the body, and
a thin, flexible wall adjacent a bottom of the recess, said wall being adapted to deform outwardly into the recess to provide a cavity between the pad body and the skin for holding a volume of medication which has been pre-applied to the skin.

2. A body pad as set forth in claim 2 further comprising an opening in the pad body for receiving a digit such a toe.

3. A body pad as set forth in claim 1 wherein the pad body is configured to permit stretching for use of the pad as a toe straightener.

4. A body pad as set forth in claim 1 configured for treating a bunion, wherein said pad body is elongate and has a distal end, a proximal end and opposite sides, said recess being located generally toward the proximal end of the pad body, and a toe opening generally toward the distal end of the pad body for receiving a first toe of the foot.

5. A body pad as set forth in claim 4 wherein a portion of the pad body between said toe opening and the distal end of the body forms a toe spacer having a thickness of at least 0.1 in. for maintaining a desired spacing between the first and second toes.

6. A body pad as set forth in claim 4 wherein said toe spacer has a concave distal edge for contact with an interdigital surface of the foot.

7. (canceled)

8. (canceled)

9. A toe crest pad comprising
a pad body having a proximal end, a distal end and opposite sides,
only one opening in the pad body sized for receiving one or more toes,
said pad body having a distal end margin forming an anti-flex distal portion extending from adjacent one side of the pad body to adjacent the opposite side of the pad body,
said anti-flex distal portion being configured to apply an upward force to the bottom of the foot generally at the proximal phalangeal joint of each of said or more toes when the pad is applied to the foot, and
said pad body having a proximal end margin forming an anti-flex proximal portion extending from adjacent one side of the body to adjacent the opposite side of the pad body,
said anti-flex proximal being configured to apply a downward force to the top of the foot generally at the proximal phalangeal joint of each of said or more toes when the pad is applied to the foot.

10. The toe crest pad of claim 9 wherein said anti-flex distal portion of the body pad is thicker than the anti-flex proximal portion of the body pad.

11. The toe crest pad of claim 9 further comprising a pull tab extending in a proximal direction from the proximal end of the pad body for assisting in applying the toe crest pad to the foot.

12. The toe crest pad of claim 9 wherein a bottom face of the anti-flex proximal portion is generally co-planar with a bottom face of the pull tab when the pad is in a relaxed condition.

13. The toe crest pad of claim 9 wherein said bottom face of the anti-flex proximal portion is offset below the top face of the anti-flex distal portion.

14. The toe crest pad of claim 9 wherein said anti-flex proximal portion has a concave proximal face proximal of said top face.

15. (canceled)

16. (canceled)

17. A toe crest pad comprising
an elongate pad body having a substantially planar inner surface and an outer surface,
a hinge extending transversely of the pad at a location between opposite ends of the body,
first and second openings in the pad body on opposite sides of the hinge adapted to align with one another when the pad body is folded about the hinge to a folded condition for application to a foot,
first and second ribs on the outer surface of the pad body extending transversely of the pad body immediately adjacent the hinge on opposite sides of the hinge and between the first and second openings,
said first and second ribs having generally flat faces which combine to provide a substantially flat surface when the pad is in said folded condition, and
a third rib extending transversely of the pad body on the outside surface of the body adjacent a proximal end of the pad body,
said third rib having a generally semi-circular transverse cross section with a curved outer surface,
the pad being adapted to be placed on the foot with one or more toes received in said aligned openings, with the first and second ribs positioned on the underside of the foot with their flat faces combining to provide a substantially flat downwardly facing surface, and with the third rib positioned on the upper side of the foot with its curved outer surface facing upward.

18. The body pad of claim 1 wherein said pad body is made of a gel-like material, and a backing layer on the inner surface of the pad body adapted to be removed prior to adhering the body pad to said skin.

19. The body pad of claim 18 further comprising a layer of antibacterial material on said outer surface of the pad body.

20. The toe crest pad of claim 9 wherein said pad body is made of a gel-like material, and a backing layer on the inner surface of the pad body adapted to be removed prior to adhering the body pad to said skin.

21. The toe crest pad of claim 20 further comprising a layer of antibacterial material on said outer surface of the pad body.

22-26. (canceled)
